SEQUENCE LISTING (45 sequences)

SEQ ID NO: 1 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 2 - protein, 790 aa, Mycobacterium tuberculosis
MTQTPDREKALELAVAQIEKSYGKGSVMRLGDEARQPISVIPTGSIALDVALGIGGLPRGRVIEIYGPESSGKTTVALHAVANAQAAGGVAAFIDAEHALDPDYAKKLGVDTDSLLVSQPDTGEQALEIADMLIRSGALDIVVIDSVAALVPRAELEGEMGDSHVGLQARLMSQALRKMTGALNNSGTTAIFINQLRDKIGVMFGSPETTTGGKALKFYASVRMDVRRVETLKDGTNAVGNRTRVKVVKNKCLAEGTRIFDPVTGTTHRIEDVVDGRKPIHVVAAAKDGTLHARPVVSWFDQGTRDVIGLRIAGGAIVWATPDHKVLTEYGWRAAGELRKGDRVAQPRRFDGFGDSAPIPADHARLLGYLIGDGRDGWVGGKTPINFINVQRALIDDVTRIAATLGCAAHPQGRISLAIAHRPGERNGVADLCQQAGIYGKLAWEKTIPNWFFEPDIAADIVGNLLFGLFESDGWVSREQTGALRVGYTTTSEQLAHQIHWLLLRFGVGSTVRDYDPTQKRPSIVNGRRIQSKRQVFEVRISGMDNVTAFAESVPMWGPRGAALIQAIPEATQGRRRGSQATYLAAEMTDAVLNYLDERGVTAQEAAAMIGVASGDPRGGMKQVLGASRLRRDRVQALADALDDKFLHDMLAEELRYSVIREVLPTRRARTFDLEVEELHTLVAEGVVVHNCSPPFKQAEFDILYGKGISREGSLIDMGVDQGLIRKSGAWFTYEGEQLGQGKENARNFLVENADVADEIEKKIKEKLGIGAVVTDDPSNDGVLPAPVDF

SEQ ID NO: 3 - protein, 348 aa, Bacillus subtilis
MSDRQAALDMALKQIEKQFGKGSIMKLGEKTDTRISTVPSGSLALDTALGIGGYPRGRIIEVYGPESSGKTTVALHAIAEVQQQGGQAAFIDAEHALDPVYAQKLGVNIEELLLSQPDTGEQALEIAEALVRSGAVDIVVVDSVAALVPKAEIEGDMGDSHVGLQARLMSQALRKLSGAINKSKTIAIFINQIREKVGVMFGNPETTPGGRALKFYSSVRLEVRRAEQLKQGNDVMGNKTKIKVVKNKVAPPFRTAEVDIMYGEGISKEGEIIDLGTELDIVQKSGSWYSYEEERLGQGRENAKQFLKENKDIMLMIQEQIREHYGLDNNGVVQQQAEETQEELEFEE

SEQ ID NO: 4 - protein, 356 aa, Yersinia pseudotuberculosis
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYAKKLGVDIDNLLCSQPDTGEQALEICDALTRSGAVDVIIVDSVAALTPKAEIEGEIGDSHMGLAARMMSQAMRKLAGNLKNANTLLIFINQIRMKIGVMFGNPETTTGGNALKFYASVRLDIRRIGAVKDGDVVVGSETRVKVVKNKIAAPFKQAEFQILYGEGININGELVDLGVKHKLIEKAGAWYSYNGDKIGQGKANASNYLKENPAIAAELDKKLREMLLNGGNGEQPVAAATAEFADGADETNEEF

SEQ ID NO: 5 - protein, 11 aa, Artificial Sequence
  TAT cell penetrating polypeptide
YGRKKRRQRRR

SEQ ID NO: 6 - protein, 21 aa, Artificial Sequence
  Pep-1 cell penetrating polypeptide
KETWWETWWTEWSQPKKKRKV

SEQ ID NO: 7 - protein, 13 aa, Artificial Sequence
  MPG cell penetrating polypeptide
SVVDRVAEQDTQA

SEQ ID NO: 8 - protein, 11 aa, Artificial Sequence
  peptide linker
SGLRSRAAANT

SEQ ID NO: 9 - DNA, 1062 bp, Escherichia coli
ttaaaaatcttcgttagtttctgctacgccttcgctatcatctacagagaaatccggcgttgagttcgggttgctcagcagcaactcacgtactttcttctcgatctctttcgcggtttccgggttatctttcagccaggcagtcgcattcgctttaccctgaccgatcttctcacctttgtagctgtaccacgcgcctgctttctcgatcagcttctcttttacgcccaggtcaaccagttcgccgtagaagttgataccttcgccgtagaggatctggaattcagcctgtttaaacggcgcagcgattttgttcttcaccactttcacgcgggtttcgctacccaccacgttttcgccctctttcaccgcgccgatacgacggatgtcgagacgaacagaggcgtagaatttcagcgcgttaccaccggtagtggtttccgggttaccgaacatcacaccaattttcatacggatctggttgatgaagatcagcagcgtgttggactgcttcaggttacccgccagcttacgcatcgcctggctcatcatacgtgccgcaaggcccatgtgagagtcgccgatttcgccttcgatttccgctttcggcgtcagtgccgccacggagtcaacgacgataacgtctactgcgccagaacgcgccagggcgtcacagatttccagtgcctgctcgccggtgtccggctgggagcacagcaggttgtcgatatcgacgcccagtttacgtgcgtagattgggtccagcgcgtgttcagcatcgataaacgcacaggttttaccttcacgctgcgctgcggcgatcacctgcagcgtcagcgtggttttaccggaagattccggtccgtagatttcgacgatacggcccatcggcagaccacctgccccaagcgcgatatccagtgaaagcgaaccggtagagatggtttccacatccatggaacggtcttcacccaggcgcatgatggagcctttaccaaattgtttctcaatctggcccagtgctgccgccaacgctttctgtttgttttcgtcgatagccat

SEQ ID NO: 10 - DNA, 2373 bp, Mycobacterium tuberculosis
tcagaagtcgacgggggcgggcaggacaccgtcatttgagggatcatcggtcaccacggcaccaatgccaagcttttccttgatcttcttctcgatctcgtcagccacgtcggcgttctccaccaagaagttgcgggcattctccttgccctggccgagctgctcgccctcgtaggtgaaccaggcacccgacttgcggatgaggccctgatccacacccatgtcgatcagcgagccctccctgctgattcccttgccgtagaggatgtcgaactcggcctgcttgaaggggggcgaacagttgtgcacgacaaccccttcggcgacgagggtgtgcagttcctcgacctcgaggtcgaacgttcgtgcccgccgcgttggcagcacttctcggatcacggaatagcggagttcttccgccagcatgtcgtgcaggaatttgtcatccagggcatccgcgagcgcctgcacgcgatcccgacgaaggcggctggcacctaagacctgcttcattccaccgcgggggtccccggaagctacaccgatcatggccgcggcctcctgcgcggtcacgccgcgctcgtccagataattcagcacggcatcggtcatctctgcagccagatatgtcgcttgcgatccacgacgccgcccctgcgtggcttctggaatcgcctggataagcgcggcaccgcgcggcccccacatgggaactgactccgcgaatgccgtgacgttatccatacccgagatccggacctcgaacacttgacgtttgctctggatccgtcgaccgttgacgatgctcggccgcttctgggtcggatcgtaatctcgaacggtgctcccgacaccgaaccgcagcagcagccaatgaatctgatgcgcgagttgttcagaggtcgtcgtgtaaccgacccgaagtgccccggtctgttcccggctcacccacccgtcgctttcgaacaggccgaagagcagattgccgacaatgtcggccgcgatgtccggctcgaagaaccaattcggaatcgtcttctcccacgcgagcttgccgtagataccggcctgctgacaaaggtctgccacaccgttgcgctcaccgggtcgatgagcgatcgcgagtgagatacgcccctgcggatgggccgcgcaaccgagcgtcgcagcgattcgcgtcacgtcgtcaatgagcgcccgctgaacattgatgaagttgatcggagtcttgccccccacccaaccatccctgccatctccgatcaggtagccaagcagccgggcatgatccgccggaatcggcgcactgtcaccgaatccatcgaagcgtcgcggttgcgccaccctgtctcccttgcggagttccccggcggcacgccagccgtactctgtcagcaccttgtgatcgggtgtcgcccacacgatggcgccaccggcgatccgcaacccgatcacatcccgcgttccctggtcgaaccaggacaccacgggccgcgcatgcagcgttccgtccttggcagcagccacgacatgaataggcttgcgcccatcgacaacatcctcgatgcgatgcgttgtaccggtgaccggatcgaagatccgagtgccctctgcgaggcacttgttcttgacgaccttgacccgggtgcggttgccgaccgcgttggtaccgtccttgagcgtctcgactcgccgcacgtccatgcgcaccgacgcgtagaacttcaacgcctttccgcccgttgtcgtctcgggcgacccgaacatcactccgatcttgtcgcggagctggttgatgaagatcgccgtggtgcccgaattattcagcgcgccggtcattttccgcagcgcctggctcatcagccgggcctgcagcccgacgtggctgtcgcccatctcgccttcgagctccgcgcgcggcaccagcgccgccaccgagtcgatcaccacgatgtcaagcgcacccgagcggatcagcatgtcggcgatctcgagtgcctgttccccggtgtccggctggctgaccagcagcgaatcggtgtcgacaccgagcttcttggcatagtccggatccagcgcgtgctcggcgtcgatgaacgccgcaacaccaccggcggcctgagcgttggccaccgcgtgcagcgccacggtggtcttacccgacgactccgggccgtatatctctatcacccggccacgcggcaggccgccaatgcccagggccacgtctagtgcgatggatccggtcggaatgaccgaaatcggctgacgcgcctcgtcgccgaggcgcatcaccgaacctttgccgtaactcttctcgatctgggccactgccagctcgagcgccttttcccgatcgggggtctgcgtcat

SEQ ID NO: 11 - DNA, 1047 bp, Bacillus subtilis
atgagtgatcgtcaggcagccttagatatggctcttaaacaaatagaaaaacagttcggcaaaggttccattatgaaactgggagaaaagacagatacaagaatttctactgtaccaagcggctccctcgctcttgatacagcactgggaattggcggatatcctcgcggacggattattgaagtatacggtcctgaaagctcaggtaaaacaactgtggcgcttcatgcgattgctgaagttcagcagcagggcggacaagccgcgtttatcgatgcggagcatgcgttagatccggtatacgcgcaaaagctcggtgttaacatcgaagagcttttactgtctcagcctgacacaggcgagcaggcgcttgaaattgcggaagcattggttcgaagcggggcagttgacattgtcgttgtcgactctgtagccgctctcgttccgaaagcggaaattgaaggcgacatgggagattcgcatgtcggtttacaagcacgcttaatgtctcaagcgcttcgtaagctttcaggggccattaacaaatcgaagacaatcgcgattttcattaaccaaattcgtgaaaaagtcggtgttatgttcgggaacccggaaacaacacctggcggccgtgcgttgaaattctattcttccgtgcgtcttgaagtgcgccgtgctgaacagctgaaacaaggcaacgacgtaatggggaacaaaacgaaaatcaaagtcgtgaaaaacaaggtggctccgccgttccgtacagccgaggttgacattatgtacggagaaggcatttcaaaagaaggcgaaatcattgatctaggaactgaacttgatatcgtgcaaaaaagcggttcatggtactcttatgaagaagagcgtcttggccaaggccgtgaaaatgcaaaacaattcctgaaagaaaataaagatatcatgctgatgatccaggagcaaattcgcgaacattacggcttggataataacggagtagtgcagcagcaagctgaagagacacaagaagaactcgaatttgaagaataa

SEQ ID NO: 12 - DNA, 1071 bp, Yersinia pseudotuberculosis
atggctattgatgagaataaacaaaaggcgttagcagcagcactgggccaaattgaaaaacaattcggtaaaggctctattatgcgccttggcgaagaccgctcaatggatgttgaaaccatctctaccggctccctttcccttgatattgcactgggggctggtggcttaccaatggggcgtatcgttgagatttatggcccagaatcatcaggtaagacgacactgacattacaggttatcgccgccgcacagcgtgaaggcaaaacgtgtgcatttatcgatgccgaacatgcccttgacccaatctatgccaagaaattgggtgtagatattgataacctattgtgttctcagccagatactggcgagcaggcactggaaatttgtgatgcgctgactcgctctggtgcggttgacgttatcatcgttgactccgtagcggcattgacaccaaaagctgaaattgaaggtgaaattggcgattctcatatgggccttgccgcgcgtatgatgagccaggctatgcgtaagctggcgggtaacctgaagaatgcgaataccttactgatttttatcaaccaaatccgcatgaaaattggcgtgatgtttggtaacccagaaaccactaccggtggcaacgctcttaaattttacgcttctgtacgtttggatatccgccgtattggtgcagtaaaagatggtgatgtggtcgtggggagtgaaacccgcgttaaagtcgttaaaaacaagattgctgcgccattcaaacaagctgaattccagatcctctacggtgaaggcattaatatcaacggtgaactggttgacttaggtgttaaacacaaactgattgagaaagctggcgcatggtatagctataacggtgataaaattggtcagggtaaagccaatgccagcaactatttaaaagaaaacccagccattgctgctgagttagataaaaaactgcgtgaaatgctacttaatggcggcaatggtgaacaacctgttgctgcggcaacagcagaattcgccgatggtgcagatgaaaccaacgaagaattttag

SEQ ID NO: 13 - DNA, 33 bp, Artificial Sequence
  nucleic acid encoding a TAT cell penetrating polypeptide
tacggcaggaagaagcggagacagcgacgaaga

SEQ ID NO: 14 - DNA, 32 bp, Artificial Sequence
  synthetic oligonucleotide primer
gcccgggaccatggctattgatgagaataaac

SEQ ID NO: 15 - DNA, 65 bp, Artificial Sequence
  synthetic oligonucleotide primer
ctctagaaataattttgtttaactttaagaaggagatatacatatggctattgatgagaataaac

SEQ ID NO: 16 - DNA, 21 bp, Artificial Sequence
  synthetic oligonucleotide primer
caatttcttggcatagattgg

SEQ ID NO: 17 - DNA, 21 bp, Artificial Sequence
  synthetic oligonucleotide primer
ccattcaaacaagctgaattc

SEQ ID NO: 18 - DNA, 69 bp, Artificial Sequence
  synthetic oligonucleotide primer
gaaaccaacgaagaatttagtggcctacgatcgcgagcagctgcgaacacgatgagtattcaacatttc

SEQ ID NO: 19 - DNA, 19 bp, Artificial Sequence
  synthetic oligonucleotide primer
cgggataataccgcgccac

SEQ ID NO: 20 - DNA, 69 bp, Artificial Sequence
  synthetic oligonucleotide primer
gaaatgttgaatactcatcgtgttcgcagctgctcgcgatcgtaggccactaaattcttcgttggtttc

SEQ ID NO: 21 - DNA, 22 bp, Artificial Sequence
  synthetic oligonucleotide primer
gcttcccggcaacaattaatag

SEQ ID NO: 22 - DNA, 20 bp, Artificial Sequence
  synthetic oligonucleotide primer
gagtaaacttggtctgacag

SEQ ID NO: 23 - DNA, 60 bp, Artificial Sequence
  synthetic oligonucleotide primer
gtgaagttgacatccaaaaaggatgttttctcgtgctgcagcccaatgcttaatcagtga

SEQ ID NO: 24 - DNA, 60 bp, Artificial Sequence
  synthetic oligonucleotide primer
cttcttcctgccgtaatggtgatggtgatggtgatggtgatggtgaagttgacatccaaa

SEQ ID NO: 25 - DNA, 47 bp, Artificial Sequence
  synthetic oligonucleotide primer
gcggccgcctatcttcgtcgctgtctccgcttcttcctgccgtaatg

SEQ ID NO: 26 - DNA, 76 bp, Artificial Sequence
  synthetic oligonucleotide primer
ggtggcggccgctctagaaataattttgtttaactttaagaaggagatatacatatggctattgatgagaataaac

SEQ ID NO: 27 - DNA, 76 bp, Artificial Sequence
  synthetic oligonucleotide primer
gtttattctcatcaatagccatatgtatatctccttcttaaagttaaacaaaattatttctagagcggccgccacc

SEQ ID NO: 28 - DNA, 42 bp, Artificial Sequence
  synthetic oligonucleotide primer
cgagcagctgcgaacacgggatccgctgcagcacgagaaaac

SEQ ID NO: 29 - DNA, 42 bp, Artificial Sequence
  synthetic oligonucleotide primer
gttttctcgtgctgcagcggatcccgtgttcgcagctgctcg

SEQ ID NO: 30 - DNA, 1055 bp, Artificial Sequence
  nucleic acid sequence encoding a wild type GFP
atggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccattgccttcgccagatcccgcgctcagtcgtccaattctgccgtggacggcaccgccggacccggctccaccggatctcgctaagaattcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggacttt

SEQ ID NO: 31 - DNA, 1051 bp, Artificial Sequence
  nucleic acid sequence encoding a loss-of-function GFP
atggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacggggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccattgccttcgccagatcccgcgctcagtcgtccaattctgccgtggacggcaccgccggacccggctccaccggatctcgctaagaattcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggacttt

SEQ ID NO: 32 - DNA, 60 bp, Artificial Sequence
  synthetic oligonucleotide to correct a GFP cDNA frame shift deletion
ggcgccctgaccttcagcccctacctgctgagccacgtgatgggctacggcttctaccac

SEQ ID NO: 33 - DNA, 81 bp, Artificial Sequence
  synthetic oligonucleotide to correct a GFP cDNA frame shift deletion
aacaagatgaagagcaccaaaggcgccctgaccttcagcccctacctgctgagccacgtgatgggctacggcttctaccac

SEQ ID NO: 34 - DNA, 102 bp, Artificial Sequence
  synthetic oligonucleotide to correct a GFP cDNA frame shift deletion
cccaagcagggccgcatgaccaacaagatgaagagcaccaaaggcgccctgaccttcagcccctacctgctgagccacgtgatgggctacggcttctaccac

SEQ ID NO: 35 - DNA, 60 bp, Artificial Sequence
  synthetic oligonucleotide to correct a GFP cDNA frame shift deletion
ccgctggggtaggtgccgaagtggtagaagccgtagcccatcacgtggctcagcaggtag

SEQ ID NO: 36 - DNA, 81 bp, Artificial Sequence
  synthetic oligonucleotide to correct a GFP cDNA frame shift deletion
tgcaggaaggggttctcgtagccgctggggtaggtgccgaagtggtagaagccgtagcccatcacgtggctcagcaggtag

SEQ ID NO: 37 - DNA, 102 bp, Artificial Sequence
  synthetic oligonucleotide to correct a GFP cDNA frame shift deletion
tagccgccgttgttgatggcgtgcaggaaggggttctcgtagccgctggggtaggtgccgaagtggtagaagccgtagcccatcacgtggctcagcaggtag

SEQ ID NO: 38 - DNA, 671 bp, Mus musculus
aagctttatccccgctgccatcatggttcgaccattgaactgcatcgtcgccgtgtcccaagatatggggattggcaagaacggagacctaccctggcctccgctcaggaacgagtggaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatct

SEQ ID NO: 39 - DNA, 669 bp, Artificial Sequence
  nucleic acid sequence encoding a loss-of-function mouse dihydrofolate reductase
aagctttatccccgctgccatcatggttcgaccattgaactgcatcgtcgccgtgtcccaagatatggggattggcaagaacggagacctaccctggcctccgctcaggaacgagtggaagtacttccaaagaaaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatct

SEQ ID NO: 40 - DNA, 60 bp, Artificial Sequence
  synthetic oligonucleotide to correct a DHFR cDNA frame shift deletion
tggcctccgctcaggaacgagtggaagtacttccaaagaatgaccacaacctcttcagtg

SEQ ID NO: 41 - DNA, 81 bp, Artificial Sequence
  synthetic oligonucleotide to correct a DHFR cDNA frame shift deletion
ggcaagaacggagacctaccctggcctccgctcaggaacgagtggaagtacttccaaagaatgaccacaacctcttcagtg

SEQ ID NO: 42 - DNA, 102 bp, Artificial Sequence
  synthetic oligonucleotide to correct a DHFR cDNA frame shift deletion
gtgtcccaagatatggggattggcaagaacggagacctaccctggcctccgctcaggaacgagtggaagtacttccaaagaatgaccacaacctcttcagtg

SEQ ID NO: 43 - DNA, 60 bp, Artificial Sequence
  synthetic oligonucleotide to correct a DHFR cDNA frame shift deletion
tcaccacattctgtttaccttccactgaagaggttgtggtcattctttggaagtacttcc

SEQ ID NO: 44 - DNA, 81 bp, Artificial Sequence
  synthetic oligonucleotide to correct a DHFR cDNA frame shift deletion
accaggttttcctacccataatcaccacattctgtttaccttccactgaagaggttgtggtcattctttggaagtacttcc

SEQ ID NO: 45 - DNA, 102 bp, Artificial Sequence
  synthetic oligonucleotide to correct a DHFR cDNA frame shift deletion
gattcttctcaggaatggagaaccaggttttcctacccataatcaccacattctgtttaccttccactgaagaggttgtggtcattctttggaagtacttcc

(19) United States
(12) Patent Application Publication
(10) Pub. No.: US 2021/0206814 A1
(43) Pub. Date: Jul. 8, 2021

(54) METHODS AND MATERIALS FOR GENE EDITING

(71) Applicant: Mayo Foundation for Medical Education and Research, Rochester, MN (US)

(72) Inventor: Xiu-Bao Chang, Scottsdale, AZ (US)

(73) Assignee: Mayo Foundation for Medical Education and Research, Rochester, MN (US)

(21) Appl. No.: 16/754,898

(22) PCT Filed: Oct. 12, 2018
(86) PCT No.: PCT/US2018/055677

§ 371 Date: Apr. 9, 2020

Related U.S. Application Data
(60) Provisional application No. 62/627,729, filed on Feb. 7, 2018.
(60) Provisional application No. 62/571,457, filed on Oct. 12, 2017.

(51) Int. Cl. C07K 14/195; C07K 16/24; C12N 15/90

(52) U.S. Cl. CPC C07K 14/195; C07K 16/24; C12N 15/907; A61K 38/00; C07K 2319/40; C07K 2319/60; C07K 2319/10; C07K 2319/30

(57) ABSTRACT

This document relates to methods and materials for gene editing. For example, methods and materials for using a RecA polypeptide fused to a cell penetrating peptide to edit (e.g., correct) a gene are provided.

### CROSS-REFERENCE TO RELATED APPLICATIONS

This application claims the benefit of U.S. Patent Application Ser. No. 62/571,457, filed on Oct. 12, 2017, and U.S. Patent Application Ser. No. 62/627,729, filed on Feb. 7, 2018. The entire contents of which are hereby incorporated by reference.

### BACKGROUND

### 1. Technical Field

This document relates to methods and materials involved in gene editing. For example, this document provides methods and materials for using a RecA polypeptide fused to a cell penetrating peptide (CPP) to edit (e.g., correct) a gene.

### 2. Background Information

Many genetic disorders, such as color blindness (see, e.g., Nathans et al., 1989 Science 245:831-838; Weitz et al., 1992 Am J Hum Genet 50:498-507; Winderickx et al., 1992 Nat Genet 1:251-256; and Mackey, 1994 Eye (Lond) 8(Pt 4):431-436); cystic fibrosis (see, e.g., Kerem et al., 1989 Science 245:1073-1080; and Bobadilla et al., 2002 Hum Mutat 19:575-606); haemochromatosis (see, e.g., Feder et al., 1996 Nat Genet 13:399-408; and Pietrangelo et al., 1999 N Engl J Med 341:725-732); haemophilia (see, e.g., Gitschier et al., 1985 Nature 315:427-430; Rees et al., 1985 Nature 316:643-645; Bentley et al., 1986 Cell 45:343-348; Davis et al., 1987 Blood 69:140-143; Youssoufian et al., 1986 Nature 324:380-382; Diuguid et al., 1986 Proc Natl Acad Sci USA 83:5803-5807; and Gitschier et al., 1986 Science 232:1415-1416); phenylketonuria (see, e.g., DiLella et al., 1987 Nature 327:333-336; and Lyonnet et al., 1989 Am J Hum Genet 44:511-517); polycystic kidney disease (see, e.g., Bisceglia et al., 2006 Adv Anat Pathol 13:26-56; and Audrezet et al., 2012 Hum Mutat 33:1239-1250); sickle-cell disease (see, e.g., ghr.nim.nih.gov/condition/sickle-cell-disease); and some of the duchenne muscular dystrophy (see, e.g., Aartsma-Rus et al., 2006 Muscle Nerve 34:135-144), are caused by small deletion/insertion or simple point mutations. For example, a deletion of three nucleotide (nt) coding for phenylalanine at position of 508 (ΔF508) in the cystic fibrosis transmembrane conductance regulator (CFTR) or ATP-binding cassette transporter C7 (ABCC7) gene, the most common mutation in cystic fibrosis, results in thermolability and mis-folding of the CFTR/ABCC7 ion channel protein on the apical membrane of epithelial cells (see, e.g., Cheng et al., 1990 Cell 63:827-834; and Denning et al., 1992 Nature 358:761-764) and causes cystic fibrosis. Such disease-causing mutations can potentially be corrected by homology-directed recombination (HDR).

However, HDR is a complex processing of orchestrated reactions involving multiple factors. In addition, presynaptic single stranded DNA (ssDNA) invasion (searching for homologous sequences) plays a crucial role for initiation of the HDR. The greatest challenge in HDR-mediated gene correction is the creation of recombinogenic DNA ends near the mutation site. Development of the clustered regularly interspaced short palindromic repeats (CRISPR)/CRISPR-associated protein 9 (Cas9) system provides a mean to cut the DNA (e.g., by making a double strand DNA (dsDNA) break) near the mutation site (see, e.g., Ramirez et al., 2008 Nat Methods 5:374-375; Maeder et al., 2008 Mol Cell 31:294-301; Boch, 2011 Nat Biotechnol 29:135-136; Jinek et al., 2012 Science 337:816-821; Pennisi, 2013 Science 341:833-836; Ran et al., 2013 Nature protocols 8:2281-2308; Ran et al., 2013 Cell 154:1380-1389; and Cong et al., 2013 Science 339:819-823). Unfortunately, non-homologous end-joining (NHEJ), albeit without ensuring restoration of the DNA sequence around the break site, plays a dominant role over HDR for any dsDNA break repair in mammalian cells (see, e.g., Fu et al., 2013 Nat Biotechnol 31:822-826; Mali et al., 2013 Nat Biotechnol 31:833-838; and Hsu et al., 2013 Nat Biotechnol 31:827-832), meaning that the efficiency of the HDR-mediated repair of the mutation near the CRISPR/Cas9-gRNA cutting site could be low. In addition, the modifications at the break site, including a few nucleotides insertion (see, e.g., Roth et al., 1989 Mol Cell Biol 9(7):3049-3057; and Chang et al., 1987 Proc Natl Acad Sci USA 84:4959-4963) and/or deletion (see, e.g., Smithies et al., 1985 Nature 317:230-234), may cause deleterious mutations, suggesting that mutations introduced by CRISPR/Cas9 system may dominate the HDR of the disease-causing mutations. In fact, the frequency of mutations introduced by guideRNA complementary to the target DNA is significantly higher than the gene-correction mediated by HDR (see, e.g., Thomas et al., 1986 Cell 44:419-428; and Xu et al., 2017 Mol Ther Nucleic Acids 16:429-438). In addition, the random dsDNA break insertions, such as CRISPR/Cas9 DNA or donor DNA insertion into chromosomes, and/or off-target modifications may also cause mutations that affect normal cell functions. Furthermore, it has been reported that unexpected mutations occurred after CRISPR-Cas9-mediated genome editing in vivo (see, e.g., Roth et al., 1989 Mol Cell Biol 9:3049-3057; and Schaefer et al., 2017 Nat Methods 14(6):547-548), suggesting that safety is a very important issue in CRISPR/Cas9 mediated gene correction. Thus, a safer technology is critically needed in the design of strategies to correct mutations in genetic disease.

### SUMMARY

This document relates to methods and materials for gene editing. For example, this document provides methods and materials for using a RecA polypeptide fused to a cell penetrating peptide (CPP) to edit (e.g., correct) a nucleic acid sequence (e.g., a coding sequence such as a gene) within a cell. In some cases, the methods and materials provided herein can be used to correct a nucleic acid sequence containing one or more mutations such as deletions/insertions and/or point mutations. For example, a RecA polypeptide fused to a CPP can be used insert/delete a nucleic acid sequence (e.g., a coding sequence such as a gene) within a cell to correct a nucleic acid sequence containing one or more mutations such as deletions/insertions and/or point mutations. In some cases, the methods and materials provided herein can be used to treat a mammal having a genetic disease or genetic condition (e.g., a monogenetic disease or a monogenetic condition) caused, at least in part, by one or more mutations such as a deletion/insertion and/or a point mutation in a nucleic acid sequence (e.g., a coding sequence such as a gene) within a cell. For example, a RecA polypeptide fused to a CPP can be used insert a nucleic acid sequence (e.g., a coding sequence such as a gene) within a cell of a mammal to correct a nucleic acid sequence containing one or more mutations such as deletions/insertions and/or point mutations in the cell to treat the mammal.

In general, one aspect of this document features fusion proteins including a RecA polypeptide and CPP. The RecA polypeptide can include an amino acid sequence set forth in SEQ ID NO:1, SEQ ID NO:2, SEQ ID NO:3, or SEQ ID NO:4. The RecA polypeptide can be at the N-terminus of the fusion protein. The CPP can be a trans-activating transcriptional activator (TAT) peptide sequence, a Pep-1 peptide sequence, or a MPG peptide sequence. When the CPP is a TAT peptide, the TAT peptide sequence can include the amino acid sequence YGRKKRRQRRR (SEQ ID NO:5). When the CPP is a Pep-1 peptide, the Pep-1 peptide sequence can include the amino acid sequence KETWWETWWTEWSQPKKKRKV (SEQ ID NO:6). When the CPP is a MPG peptide, the MPG peptide sequence can include the amino acid sequence SVVDRVAEQDTQA (SEQ ID NO:7). The CPP can be at the C-terminus of the fusion protein. The fusion protein further also can include a peptide linker (e.g., present between the RecA polypeptide and the CPP). The peptide linker can be a peptide sequence including SGLRSRAAANT (SEQ ID NO:8), one or more alanine residues, one or more glycine residues, or combinations thereof. The fusion protein also can include a peptide tag. The peptide tag can include an antibody epitope (e.g., a multidrug resistance protein 1 (MRP1) antibody epitope). The peptide tag can include a fluorescent protein (e.g., a green fluorescent protein GFP)). The fusion protein can include both a MRP1 antibody epitope and a GFP.

In another aspect, this document features fusion proteins including, from N-terminus to C-terminus, a RecA polypeptide, a linker, a first tag, a second tag, and a CPP. For example, the fusion protein can include, from N-terminus to C-terminus, a RecA polypeptide including the amino acid sequence set forth in SEQ ID NO:4, an L1 linker, a MRP1 antibody epitope for a first tag, ten histidine residues for a second tag, and a TAT peptide including the amino acid sequence YGRKKRRQRRR (SEQ ID NO:5) for a CPP.

In another aspect, this document features fusion proteins including, from N-terminus to C-terminus, a RecA polypeptide, a first linker, a green fluorescent protein, a second linker, a first tag, a second tag, and a CPP. For example, the fusion protein can include, from N-terminus to C-terminus, a RecA polypeptide including the amino acid sequence set forth in SEQ ID NO:4, an L1 linker as a first linker, 2 alanine residues as a second linker, a MRP1 antibody epitope as a first tag, ten histidine residues as a second tag, and a TAT peptide including the amino acid sequence YGRKKRRQRRR (SEQ ID NO:5) for a CPP.

In another aspect, this document features nucleic acid constructs encoding a fusion protein including a RecA polypeptide and CPP. The nucleic acid construct can include a nucleic acid sequence encoding a RecA polypeptide. The nucleic acid sequence encoding a RecA polypeptide can include a nucleic acid sequence set forth in SEQ ID NO:9, SEQ ID NO:10, SEQ ID NO:11, or SEQ ID NO:12. The nucleic acid construct can include a nucleic acid sequence encoding a CPP. The nucleic acid sequence encoding said CPP can include a nucleic acid sequence set forth in SEQ ID NO:13

In another aspect, this document features nucleoprotein filaments including one or more fusion proteins including a RecA polypeptide and CPP, and a single stranded oligonucleotide, where the single stranded oligonucleotide can hybridize to a target sequence. The target sequence can have one or more mutations, and the single stranded oligonucleotide can include a corrected nucleic acid sequence.

In another aspect, this document features methods for editing the genome of a cell. The methods can include, or consist essentially of, contacting a cell with a fusion protein including a RecA polypeptide and CPP; and a single stranded oligonucleotide, where the single stranded oligonucleotide can hybridize to a target sequence within the cell having one or more mutations, and where the single stranded oligonucleotide includes a corrected nucleic acid sequence. The cell can be a prokaryotic cell. The cell can be a eukaryotic cell. The eukaryotic cell can be a mammalian cell.

In another aspect, this document features methods for treating a mammal having a monogenetic disease. The methods can include, or consist essentially of, contacting a cell in a mammal having a monogenetic disease with a fusion protein including a RecA polypeptide and CPP and a single stranded oligonucleotide, where the single stranded oligonucleotide can hybridize to a target sequence in a genome within the cell, where the target sequence includes a nucleic acid sequence having one or more disease-causing mutations, and where the single stranded oligonucleotide includes a corrected nucleic sequence. The mammal can be a human. The monogenetic disease can be color blindness, cystic fibrosis, haemochromatosis, haemophilia, phenylketonuria, polycystic kidney disease, Tay-Sachs disease, Huntington's disease, Marfan syndrome, sickle-cell disease, duchenne muscular dystrophy, or cancer. The fusion protein can include, from N-terminus to C-terminus, a RecA polypeptide, a linker, a first tag, a second tag, and a CPP. For example, the fusion protein can include, from N-terminus to C-terminus, a RecA polypeptide including the amino acid sequence set forth in SEQ ID NO:4, an L1 linker, a MRP1 antibody epitope as a first tag, ten histidine residues as a second tag, and a TAT peptide including the amino acid sequence YGRKKRRQRRR (SEQ ID NO:5) as a CPP. The fusion protein can include, from N-terminus to C-terminus, a RecA polypeptide, a first linker, a green fluorescent protein, a second linker, a first tag, a second tag, and a CPP. For example, the fusion protein can include, from N-terminus to C-terminus, a RecA polypeptide including the amino acid sequence set forth in SEQ ID NO:4, an L1 linker as a first linker, GFP, 2 alanine residues as a second linker, a MRP1 antibody epitope as a first tag, ten histidine residues as a second tag, and a TAT peptide including the amino acid sequence YGRKKRRQRRR (SEQ ID NO:5) as a CPP.

In another aspect, this document features methods for detecting HDR mediated gene correction in a cell having a modified nucleic acid sequence, where the modified nucleic acid sequence can encode a polypeptide having a loss-of-function mutation. The methods can include, or consist essentially of, contacting a cell having a modified nucleic acid sequence with a fusion protein including a RecA polypeptide and CPP, and a single stranded oligonucleotide, where the single stranded oligonucleotide can hybridize to the modified nucleic acid sequence, where the single stranded oligonucleotide includes a corrected nucleic acid sequence, and where the corrected nucleic acid, in the presence of HDR, can replace the modified nucleic acid sequence and can encode a functional polypeptide; such that detection of the functional polypeptide indicates the present of HDR in the cell. The cell can be a eukaryotic cell. The cell can be a human cell. The modified nucleic acid sequence can encode a reporter polypeptide having a loss-of-function mutation, and detection of the reporter function can indicate the present of HDR in the cell. The reporter polypeptide can be GFP, the modified nucleic acid sequence encoding a GFP having a loss-of-function mutation can include the sequence set forth in SEQ ID NO:31, and the single stranded oligonucleotide including an insertion can include a sequence set forth in SEQ ID NO:32, SEQ ID NO:33, SEQ ID NO:34, SEQ ID NO:35, SEQ ID NO:36, or SEQ ID NO:37. The reporter polypeptide can be a dihydrofolate reductase (DHFR) polypeptide, the modified nucleic acid sequence encoding a DHFR having a loss-of-function mutation can include the sequence set forth in SEQ ID NO:39, and the single stranded oligonucleotide including an insertion can include a sequence set forth in SEQ ID NO:40, SEQ ID NO:41, SEQ ID NO:42, SEQ ID NO:43, SEQ ID NO:44, or SEQ ID NO:45.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B are diagrams showing exemplary RecA-CPP fusion proteins. FIG. 1A shows the design of a shorter version of a RecA-CPP fusion protein (RecA-CPP) containing a RecA polypeptide, a linker (L1), a first tag (Tag1), a second tag (Tag2), and a CPP. FIG. 1B shows the design of a longer version of a RecA-CPP fusion protein (RecA-GFP-CPP) containing a RecA polypeptide, a linker (L1), green fluorescent protein (GFP), a second linker (L2), a first tag (Tag1), a second tag (Tag2), and a CPP.

FIG. 2 is a schematic diagram showing an exemplary ssDNA:RecA-GFP-CPP fusion protein mediated transfection to correct disease-causing gene mutations.

FIGS. 3A and 3B show that RecA-CPP fusion protein expressed in bacteria is in the soluble fraction. FIG. 3A is a representative western blot (100 μg protein per lane) of the shorter RecA-CPP fusion protein probed with multidrug resistance protein 1 (MRP1) mAb 42.4. FIG. 3B is a representative western blot (100 μg protein per lane) of the longer RecA-GFP-CPP fusion protein probed with MRP1 mAb 42.4.

FIGS. 4A and 4B show that bacterial growth is completely inhibited by the addition of IPTG at 37° C. FIG. 4A shows that, after transformation of the DL21 competent cells with the shorter version of the RecA-CPP fusion construct in pET32a vector, the cells were plated out on plates with 100 μg/mL ampicillin (the plate on the left) or with 100 μg/mL ampicillin and 0.25 mM IPTG (the plate on the right). FIG. 4B shows that, after transformation of the DL21 competent cells with the longer version of the RecA-GFP-CPP fusion construct in pET32a vector, the cells were plated out on plates with 100 μg/mL ampicillin (the plate on the left) or with 100 μg/mL ampicillin and 0.25 mM IPTG (the plate on the right).

FIGS. 5A and 5B show the expression of RecA-CPP fusion proteins in BHK cells. FIG. 5A is a representative western blot (100 μg protein per lane) showed that majority of the shorter RecA-CPP fusion protein expressed in BHK cells is in soluble fraction. FIG. 5B is a representative western blot (100 μg protein per lane) showed that majority of the longer RecA-GFP-CPP fusion protein expressed in BHK cells is also in soluble fraction.

FIG. 6 contains a graph showing that expression of RecA-CPP fusion protein in BHK cells significantly inhibited cell growth. 10,000 cells were plated out on day 0 and counted after 3 days incubation at 37° C. The numbers of cells, after 3 days incubation, were: 236,667±25,403 (BHK); 81,500±12,817 (RecA-GFP-CPP); and 96,300±12,817 (RecA-CPP). * indicates that the P value is 0.2302; ***, 0.0010; ****, 0.0007.

FIG. 7 contains an image of a western blot showing a comparison of the fusion proteins expressed in bacteria and in BHK cells. The representative western blot (100 μg protein per lane), probed with MRP1 mAb 42.4, showed that RecA-GFP-CPP or RecA-CPP expressed in BHK cells is significantly less than in DL21 bacteria cells.

FIGS. 8A-8D contain amino acid sequences of exemplary RecA polypeptides. FIG. 8A contains SEQ ID NO:1. FIG. 8B contains SEQ ID NO:2. FIG. 8C contains SEQ ID NO:3. FIG. 8D contains SEQ ID NO:4.

FIGS. 9A-9D contain nucleic acid sequences encoding exemplary RecA polypeptides. FIG. 9A contains SEQ ID NO:9. FIG. 9B contains SEQ ID NO:10. FIG. 9C contains SEQ ID NO:11. FIG. 9D contains SEQ ID NO:12.

FIGS. 10A-10B contains nucleic acid sequences encoding GFP polypeptides. FIG. 10A contains a nucleic acid sequence (SEQ ID NO:30) encoding a wild type GFP. FIG. 10B contains a nucleic acid sequence having a deletion of 4 nucleotides from 185 to 188 (TGAT) of a GFP coding sequence (SEQ ID NO:31) such that the nucleic acid sequence encodes a non-functional GFP. The Δ symbols indicate the deleted nucleotides.

FIGS. 11A-11B contains nucleic acid sequences encoding mouse dihydrofolate reductase (DHFR) polypeptides. FIG. 11A contains a nucleic acid sequence (SEQ ID NO:38) encoding a wild type DHFR. FIG. 11B contains a nucleic acid sequence having a deletion of 2 nucleotides from 135 to 136 (TG) of a DHFR coding sequence (SEQ ID NO:39) such that the nucleic acid sequence encodes a non-functional DHFR. The Δ symbols indicate the deleted nucleotides.

### DETAILED DESCRIPTION

This document provides methods and materials for gene editing. For example, this document provides methods and materials for using a RecA polypeptide fused to a cell penetrating peptide (CPP) to edit (e.g., correct) a gene. In some cases, the methods and materials provided herein can be used to correct a nucleic acid sequence (e.g., a coding sequence such as a gene) containing one or more mutations such as small deletions/insertions and/or point mutations. In some cases, the methods and materials provided herein can be used to treat a mammal having a genetic disease or genetic condition (e.g., a monogenetic disease or monogenetic condition) caused, at least in part, by one or more mutations in a nucleic acid sequence (e.g., a coding sequence such as a gene) within one or more cells in the mammal. Also provided herein are fusion proteins containing a RecA polypeptide and a CPP, nucleic acid constructs encoding a fusion protein comprising a RecA polypeptide and a CPP, and nucleoprotein filaments containing one or more (e.g., one, two, three, four, five, six, seven, eight, nine, or more) fusion proteins described herein (e.g., fusion proteins including a RecA polypeptide and a CPP) and a single stranded oligonucleotide (e.g., a ssDNA).

In some cases, the methods and materials provided herein do not cause additional mutations (e.g., mutations caused by dsDNA break-mediated insertion). For example, in some cases, the methods and materials provided herein do not include any nuclease (e.g., any sequence-specific nuclease) and/or capable of introducing a dsDNA break. Examples of nucleases capable of introducing a dsDNA break include, without limitation, zinc finger nucleases (ZFNs), transcription activator-like effector nucleases (TALENs), and CRISPR associated proteins (Cas enzymes such as Cas9). For example, in some cases, the methods and materials provided herein do not include any gene editing systems that include one or more nucleases capable of introducing a dsDNA break. Examples of gene editing systems that include one or more nucleases capable of introducing a dsDNA break include, without limitation, CRISPR/Cas systems such as a CRISPR/Cas9 system.

In some cases, the methods and materials provided herein can include HDR. For example, the methods and materials provided herein can include HDR in the absence of any dsDNA break. For example, as described herein an ABCC1/MRP1/ΔF728 model system (see, e.g., Xu et al., 2017 Mol Ther Nucleic Acids 16:429-438) for gene correction with single stranded oligonucleotides covering the 3 nucleotide-deletion site, via ssDNA-RecA-CPP nucleoprotein filaments correct the deletion mutation. This method can be used to edit genes while introducing fewer mutations than the CRISPR/Cas9 system. Since this system does not need to generate a dsDNA break near the mutation site, cas9 or any other nucleases are not needed. In addition, the single strand oligonucleotides are protected from nuclease digestion via formation of nucleoprotein filament with RecA polypeptide (see, e.g., Chen et al., 2008 Nature 453(7194):761-764; and Lieber, 2010 Annu Rev Biochem 79:181-211) both in vitro (in the presence of ATP) and in vivo. In the meantime, binding of RecA to the single stranded oligonucleotide can promote HDR (see, e.g., Chen et al., 2008 Nature 453(7194):761-764; and Lieber, 2010 Annu Rev Biochem 79:181-211). To facilitate the entry of the nucleoprotein filament into the cells, RecA can be fused with cell-penetrating peptide (CPP) (see, e.g., Chang et al., 2018 Int J Biochem Mol Biol 9:1-10). Furthermore, a reporter protein (e.g., GFP) can also be included in the fusion protein so that the transfected cells can be sorted out. Thus, recombinant proteins described herein (e.g., CPP-RecA, CPP-GFP-RecA, RecA-CPP, and RecA-GFP-CPP) can be made (e.g., from N-terminus to C-terminus) and can be used in oligonucleotide-CPP-RecA nucleoprotein complex mediated transfection to treat a mammal in need thereof (e.g., to correct a disease-causing mutation in a mammal).

This document provides fusion proteins containing a RecA polypeptide and a CPP, nucleic acid constructs encoding a fusion protein comprising a RecA polypeptide and a CPP, and nucleoprotein filaments containing one or more (e.g., one, two, three, four, five, six, seven, eight, nine, or more) fusion proteins described herein (e.g., fusion proteins including a RecA polypeptide and a CPP) and a single stranded oligonucleotide.

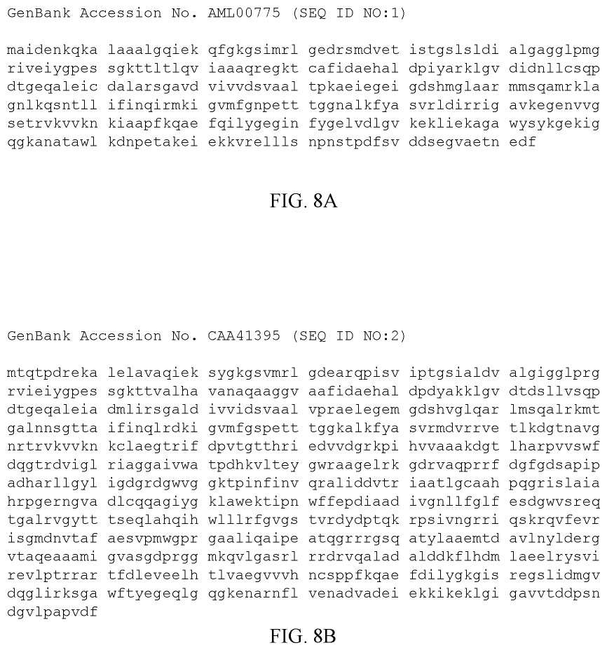

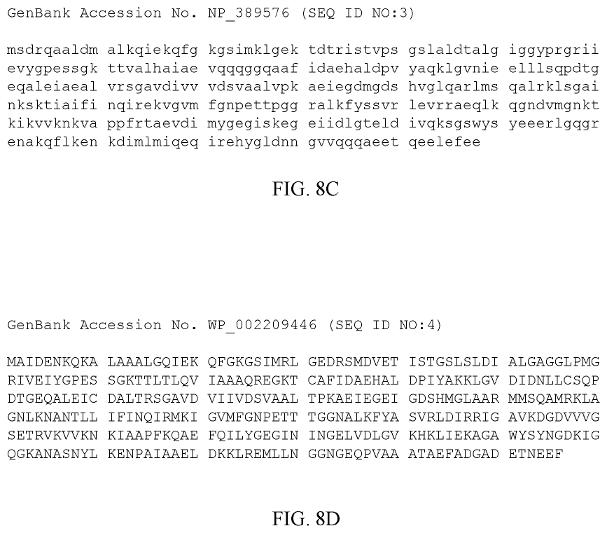

A fusion protein described herein (e.g., a fusion protein containing a RecA polypeptide and a CPP) can include any appropriate RecA polypeptide. In some cases, a RecA polypeptide can be a bacterial RecA polypeptide (e.g., Escherichia coli RecA polypeptides, Mycobacterium tuberculosis RecA polypeptides, Bacillus subtilis RecA polypeptides, and Yersinia RecA polypeptides). In some cases, a RecA polypeptide can be a mammalian homolog of a RecA polypeptide (e.g., a RAD51 polypeptide such as a human RAD51 polypeptide). Examples of RecA polypeptides include, without limitation, polypeptide sequences set forth in the National Center for Biotechnology Information (NCBI) databases at GenBank Accession No. AML00775 (Version AML00775.1), GenBank Accession No. CAA41395 (Version CAA41395.1), GenBank Accession No. NP389576 (Version NP_389576.2), GenBank Accession No. WP_002209446 (Version WP_002209446.1), and GenBank Accession No. BAA03189 (Version BAA03189.1). In some cases, RecA polypeptides can be as shown in FIG. 8. For example, a RecA polypeptide can include an amino acid sequence set forth in SEQ ID NO:1, SEQ ID NO:2, SEQ ID NO:3, or SEQ ID NO:4. In some cases, RecA polypeptides can be as described elsewhere (see, e.g., Chen et al., 2008 Nature, 453:489-4). A RecA polypeptide can be at either end of a fusion protein described herein. For example, a RecA polypeptide can be at the N-terminus of a fusion protein described herein. For example, a RecA polypeptide can be at the C-terminus of a fusion protein described herein.

In some cases, a RecA polypeptide in a fusion protein described herein (e.g., a fusion protein containing a RecA polypeptide and a CPP) can have a sequence that deviates from a wild type RecA polypeptide sequence, sometimes referred to as a variant sequence. For example, a RecA polypeptide sequence can have at least 80% sequence identity (e.g., at least 85% sequence identity, 90% sequence identity, 95% sequence identity, or at least 99% sequence identity) to SEQ ID NO:1 provided that it includes one or more amino acid additions, subtractions, or substitutions compared to SEQ ID NO:1. For example, a RecA polypeptide sequence can have at least 80% sequence identity (e.g., at least 85% sequence identity, 90% sequence identity, 95% sequence identity, or at least 99% sequence identity) to SEQ ID NO:2 provided that it includes one or more amino acid additions, subtractions, or substitutions compared to SEQ ID NO:2. For example, a RecA polypeptide sequence can have at least 80% sequence identity (e.g., at least 85% sequence identity, 90% sequence identity, 95% sequence identity, or at least 99% sequence identity) to SEQ ID NO:3 provided that it includes one or more amino acid additions, subtractions, or substitutions compared to SEQ ID NO:3. For example, a RecA polypeptide sequence can have at least 80% sequence identity (e.g., at least 85% sequence identity, 90% sequence identity, 95% sequence identity, or at least 99% sequence identity) to SEQ ID NO:4 provided that it includes one or more amino acid additions, subtractions, or substitutions compared to SEQ ID NO:4. Percent sequence identity is calculated by determining the number of matched positions in aligned polypeptide sequences, dividing the number of matched positions by the total number of aligned amino acids, respectively, and multiplying by 100. A matched position refers to a position in which identical amino acids occur at the same position in aligned sequences. The total number of aligned amino acids refers to the minimum number of RecA amino acids that are necessary to align the second sequence, and does not include alignment (e.g., forced alignment) with non-RecA sequences, such as those fused to RecA. The total number of aligned amino acids may correspond to the entire RecA sequence or may correspond to fragments of the full-length RecA sequence as defined herein. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between a RecA polypeptide and any other sequence or portion thereof using the Altschul et al. algorithm. For example, BLASTP can be used to align and compare the identity between amino acid sequences. When utilizing BLAST programs to calculate the percent identity between a RecA sequence and another sequence, the default parameters of the respective programs are used.

A fusion protein described herein (e.g., a fusion protein containing a RecA polypeptide and a CPP) can include any appropriate CPP. In some cases, a CPP can be a naturally occurring CPP. In some cases, a CPP can be an artificial CPP. In some cases, a CPP can be a synthetic CPP. Examples of CPPs include, without limitation, a TAT peptide sequence (e.g., YGRKKRRQRRR (SEQ ID NO:5)), a Pep-1 peptide sequence (e.g., KETWWETWWTEWSQPKKKRKV; SEQ ID NO:6), and a MPG peptide sequence (e.g., SVVDRVAEQDTQA; SEQ ID NO:7). In some cases, a CPP can be as described elsewhere (e.g., Okuyama et al., 2007 Nat. Methods, 4:153-9). A CPP can be at either end of a fusion protein described herein. For example, a CPP can be at the C-terminus of a fusion protein described herein. For example, a CPP can be at the N-terminus of a fusion protein described herein.

In some cases, a fusion protein described herein (e.g., a fusion protein containing a RecA polypeptide and a CPP) also can include one or more nuclear localization signal (NLS) polypeptides.

In some cases, a fusion protein described herein (e.g., a fusion protein containing a RecA polypeptide and a CPP) also can include one or more (e.g., one, two, three, or more) linkers. Examples of linkers include, without limitation, a peptide sequence including SGLRSRAAANT (SEQ ID NO:8), one or more alanine residues (e.g., 2 alanine residues), one or more glycine residues, and combinations thereof. In some cases, a linker can be as described elsewhere (e.g., Hou et al., 2009 Biochemistry, 48: 9122-9131). For example, a linker can be present between a RecA polypeptide and a CPP of a fusion protein described herein.

In some cases, a fusion protein described herein (e.g., a fusion protein containing a RecA polypeptide and a CPP) also can include one or more (e.g., one, two, three, or more) tags (e.g., detectable markers). Tags can be for detection, sorting, and/or purification of a protein. A tag can be any appropriate type of molecule (e.g., a protein tag). Examples of tags include, without limitation, fluorescent markers (e.g., GFP), epitopes (e.g., monoclonal antibody epitopes such as an MRP1 monoclonal antibody epitope), and histidine tags (e.g., a polyHis tag containing about 10 histidine residues). In cases where a fusion protein includes an MRP1 monoclonal antibody epitope, an MRP1 antibody (e.g., a monoclonal antibody such as MRP1 mAb 42.4) can be used to detect, sort, and/or purify the fusion protein (e.g., from bacterial cells and/or from mammalian cells). In some cases, a fusion protein provided herein can include a single tag. In some cases, a fusion protein provided herein can include two or more (e.g., two, three, or four) tags. A tag can be at any appropriate location within a fusion protein described herein. In some cases, a tag can be in the center (e.g., not at an end) of a fusion protein described herein. For example, a tag can be at any position between the N-terminus and C-terminus of the fusion protein. In some cases, a tag can be at an end of a fusion protein described herein. For example, a tag can be at the N-terminus of a fusion protein described herein. For example, a tag can be at the C-terminus of a fusion protein described herein.

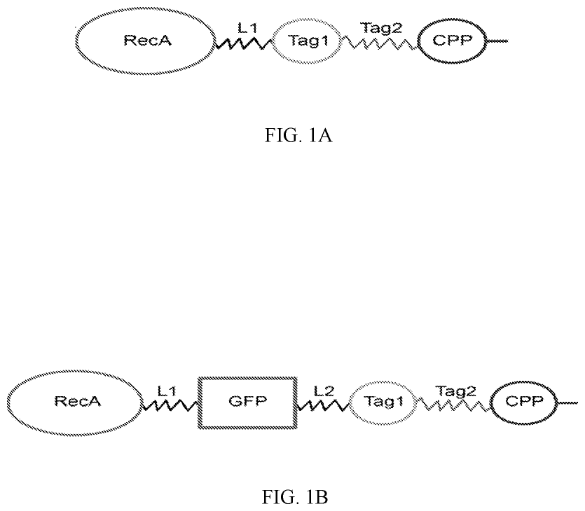

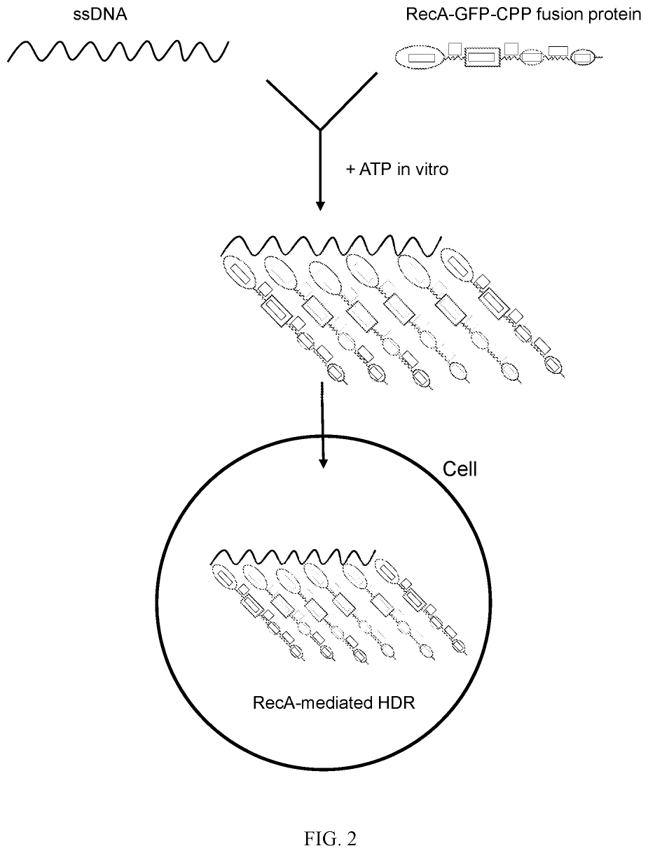

In some cases, a fusion protein can include, from N-terminus to C-terminus, a CPP and a RecA polypeptide. In some cases, a fusion protein can include, from N-terminus to C-terminus, a CPP, a GFP, and a RecA polypeptide. In some cases, a fusion protein can include, from N-terminus to C-terminus, a RecA polypeptide and a CPP. In some cases, a fusion protein can include, from N-terminus to C-terminus, a RecA polypeptide, a GFP, and a CPP. In some cases, a fusion protein can include, from N-terminus to C-terminus, a RecA polypeptide, a first linker, a GFP, a second linker, an MRP1 monoclonal antibody epitope, 10 histidine residues, and a CPP. Exemplary fusion proteins can be as shown in FIG. 1. For example, a fusion protein can include about 687 amino acids, and can contain (e.g., from N-terminus to C-terminus) a RecA, a linker (e.g., a first linker), a GFP, a linker (e.g., a second linker), an MRP1 monoclonal antibody epitope, about 10 histidine residues, and a CPP.

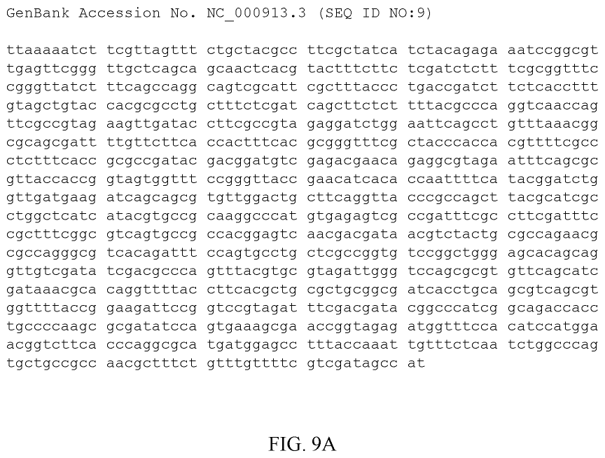

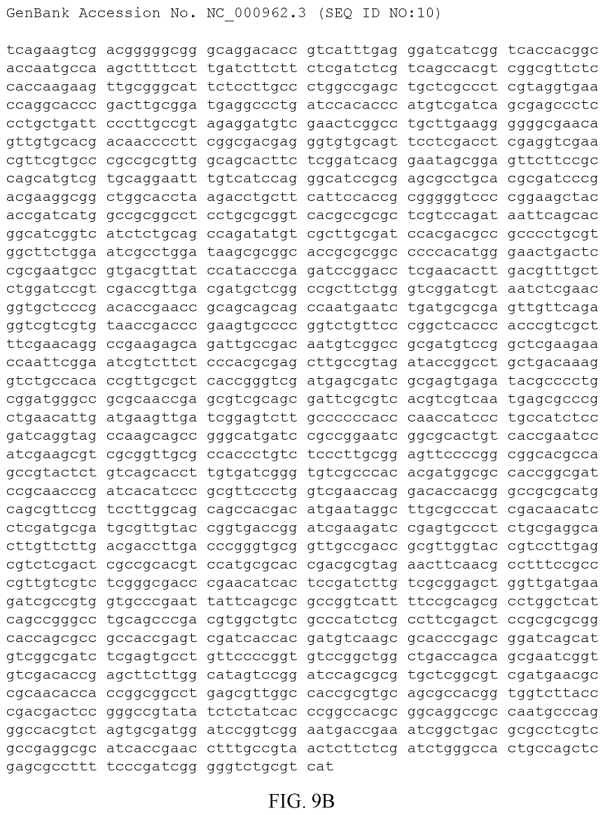

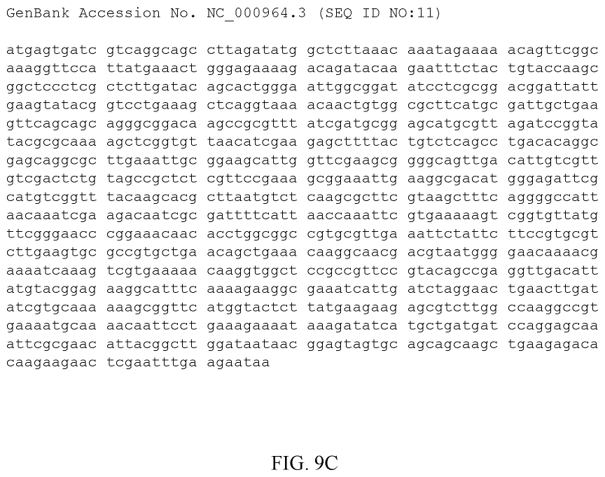

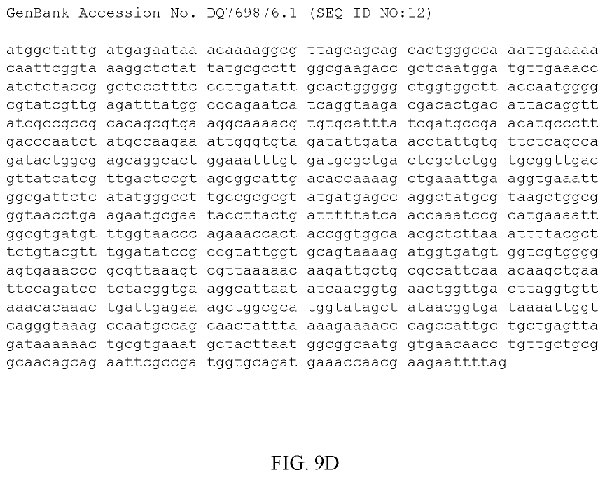

A nucleic acid construct provided herein (e.g., a nucleic acid construct encoding a fusion protein described herein (e.g., a fusion protein including a RecA polypeptide and a CPP)) can include any appropriate nucleic acid sequence encoding the fusion protein. In some cases, a nucleic acid construct can include a nucleic acid sequence (e.g., a RecA coding sequence) encoding a RecA polypeptide described herein. Examples of nucleic acid sequences encoding RecA polypeptides include, without limitation, nucleic acid sequences set forth in the NCBI databases at GenBank Accession No. NC_000913.3 (ID: 947170), GenBank Accession No. NC_000962.3 (ID: 888371), GenBank Accession No. NC_000964.3 (ID: 939497), and GenBank Accession No. DQ769876 (Version DQ769876.1). In some cases, RecA coding sequences can be as shown in FIG. 9. For example, a RecA coding sequence can include a nucleic acid sequence set forth in SEQ ID NO:9, SEQ ID NO:10, SEQ ID NO:11, or SEQ ID NO:12. In some cases, RecA coding sequences can be as described elsewhere (see, e.g., Chen et al., 2008 Nature, 453:489-4; Clone YpCD00014545 (Original Clone ID: FLH129217.01X) from the DNASU Plasmid Repository).

In some cases, a nucleic acid sequence encoding a RecA polypeptide in a nucleic acid construct described herein (e.g., a nucleic acid construct encoding a fusion protein described herein (e.g., a fusion protein containing a RecA polypeptide and a CPP)) can have a sequence that deviates from a wild type nucleic acid sequence encoding a RecA polypeptide, sometimes referred to as a variant sequence. For example, a nucleic acid sequence encoding a RecA polypeptide can have at least 80% sequence identity (e.g., at least 85% sequence identity, 90% sequence identity, 95% sequence identity, or at least 99% sequence identity) to SEQ ID NO:9 provided that it includes one or more nucleic acid additions, subtractions, or substitutions compared to SEQ ID NO:9. For example, a nucleic acid sequence encoding a RecA polypeptide can have at least 80% sequence identity (e.g., at least 85% sequence identity, 90% sequence identity, 95% sequence identity, or at least 99% sequence identity) to SEQ ID NO:10 provided that it includes one or more nucleic acid additions, subtractions, or substitutions compared to SEQ ID NO:10. For example, a nucleic acid sequence encoding a RecA polypeptide can have at least 80% sequence identity (e.g., at least 85% sequence identity, 90% sequence identity, 95% sequence identity, or at least 99% sequence identity) to SEQ ID NO:11 provided that it includes one or more nucleic acid additions, subtractions, or substitutions compared to SEQ ID NO:11. For example, a nucleic acid sequence encoding a RecA polypeptide can have at least 80% sequence identity (e.g., at least 85% sequence identity, 90% sequence identity, 95% sequence identity, or at least 99% sequence identity) to SEQ ID NO:12 provided that it includes one or more nucleic acid additions, subtractions, or substitutions compared to SEQ ID NO:12. Percent sequence identity is calculated by determining the number of matched positions in aligned nucleic acid or polypeptide sequences, dividing the number of matched positions by the total number of aligned nucleotides and multiplying by 100. A matched position refers to a position in which identical nucleotides occur at the same position in aligned sequences. The total number of aligned nucleotides refers to the minimum number of RecA nucleotides that are necessary to align the second sequence, and does not include alignment (e.g., forced alignment) with non-RecA sequences, such as those fused to RecA. The total number of aligned nucleotides may correspond to the entire RecA sequence or may correspond to fragments of the full-length RecA sequence as defined herein. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between a RecA nucleic acid molecule and any other sequence or portion thereof using the Altschul et al. algorithm. For example, BLASTN can be used to align and compare the identity between nucleic acid sequences. When utilizing BLAST programs to calculate the percent identity between a RecA sequence and another sequence, the default parameters of the respective programs are used.

A nucleic acid construct provided herein (e.g., a nucleic acid construct encoding a fusion protein described herein (e.g., a fusion protein including a RecA polypeptide and a CPP)) can include any appropriate nucleic acid sequence encoding a CPP (e.g., any appropriate CPP coding sequence). In some cases, a nucleic acid construct can include a nucleic acid sequence (e.g., a coding sequence) encoding a CPP described herein. Examples of nucleic acid sequences encoding CPPs include, without limitation, a nucleic acid sequence encoding a TAT peptide sequence (e.g., a nucleic acid sequence including the sequence TACGGCAGGAAGAAGCGGAGACAGCGACGAAGA (SEQ ID NO:13)), a nucleic acid sequence encoding a Pep-1 peptide sequence, and a nucleic acid sequence encoding a MPG peptide sequence.

A nucleoprotein filament provided herein can include one or more (e.g., one, two, three, four, five, six, seven, eight, nine, or more) fusion proteins described herein (e.g., a fusion protein including a RecA polypeptide and a CPP) and a single stranded oligonucleotide. In some cases, a nucleoprotein filament can include one or more of the same fusion protein. A RecA polypeptide of a fusion protein described herein can interact with a single stranded oligonucleotide to form a nucleoprotein filament. In some cases, a RecA polypeptide of a fusion protein described herein can protect the single stranded oligonucleotide from degradation by, for example, DNAses. In some cases, a RecA polypeptide of a fusion protein described herein can promote homologous recombination. In some cases, a CPP of a fusion protein described herein can facilitate entry of the nucleoprotein filament into a cell (e.g., a cell having one or more mutations (e.g., one or more disease-causing mutations) in a nucleic acid sequence such as coding sequence).

A nucleoprotein filament provided herein (e.g., a nucleoprotein filament including one or more fusion proteins and a single stranded oligonucleotide) can include any appropriate single stranded oligonucleotide. A single stranded oligonucleotide can include DNA, RNA, or both. For example, a single stranded oligonucleotide can be a ssDNA. A single stranded oligonucleotide can be synthetic. A single stranded oligonucleotide can (e.g., can be designed to) hybridize to a target sequence (e.g., a nucleic acid sequence (e.g., an endogenous nucleic acid sequence) having one or more mutations such as disease-causing mutations). For example, a single stranded oligonucleotide can be (e.g., can include a nucleic acid sequence that is) sufficiently complementary to a target sequence such that the single stranded oligonucleotide can hybridize to and/or recognize the target sequence. In some cases, a target sequence can be a nucleic acid sequence (e.g., a coding sequence such as a gene) that contains one or more mutations (e.g., one or more disease-causing mutations). For example, when a target sequence is a nucleic acid sequence that contains one or more nucleotides, a single stranded oligonucleotide can include an alternative nucleic acid sequence (e.g., a sequence that, via HDR, can replace (e.g., correct) nucleotides in a target sequence). In some cases, a target sequence can be a portion of a gene (e.g., an endogenous gene) that contains one or more mutations (e.g., one or more disease-causing mutations). For example, when a target sequence is a portion of a gene that contains one or more mutations, a single stranded oligonucleotide can include a corrected gene sequence (e.g., a sequence that, via HDR, can replace (e.g., correct) one or more mutations in a target sequence) such as a sequence that does not include one or more disease-causing mutations (e.g., a wild type gene sequence).

This document also provides methods for editing a nucleic acid sequence (e.g., a coding sequence such as a gene). In some cases, a method for editing a nucleic acid sequence can be used to edit a nucleic acid sequence containing one or more mutations (e.g., small deletions/insertions and/or point mutations) within a genome of a cell. For example, methods for editing a nucleic acid sequence within a cell can include contacting the cell with a fusion protein described herein (e.g., a fusion protein containing a RecA polypeptide and a CPP) and a single stranded oligonucleotide described herein (e.g., single stranded oligonucleotide capable of hybridizing to a target sequence and, optionally, including a corrected nucleic acid sequence). A cell can be any appropriate type of cell. In some cases, a cell can be a prokaryotic cell (e.g., a bacterial cell). In some cases, a cell can be a eukaryotic cell (e.g., a plant cell or a mammalian cell such as a human cell).

Any appropriate nucleic acid sequence can be edited (e.g., corrected) as described herein (e.g., by contacting a cell with a fusion protein described herein and a single stranded oligonucleotide described herein). In some cases, a nucleic acid sequence can be a coding sequence such as a gene. In some cases, a nucleic acid sequence can be an endogenous nucleic acid sequence. In some cases, a nucleic acid sequence can be within (e.g., a portion of) a gene associated with a genetic disease or genetic condition (e.g., a monogenetic disease or monogenetic condition). Examples of genes associated with a genetic disease or genetic condition include, without limitation, OPN1MW (associated with color blindness), CFTR/ABCC7 (associated with cystic fibrosis), HFE (associated with haemochromatosis), clotting factor 8 (associated with haemophilia A), clotting factor 9 (associated with haemophilia B), phenylalanine hydroxylase (associated with phenylketonuria), polycystic kidney disease 1 (PKD1; associated with polycystic kidney disease), PKD2 (associated with polycystic kidney disease), hemoglobin-Beta (associated with sickle-cell disease), Hex-A (associated with Tay-Sachs disease), huntingtin (associated with Huntington's disease), FBN1 (associated Marfan syndrome), dystrophin (associated with Duchene muscular dystrophy), and genes associated with cancers such as BRCA1, BRCA2, TP53, PTEN, MSH2, MLH1, MSH6, PMS2, EPCAM, APC, RB1, and PALB2.

A nucleic acid sequence (e.g., a coding sequence such as a gene) that can be edited as described herein can include one or more mutations. A mutation can be any appropriate type of mutation. Examples of mutations include, without limitation, deletions, insertions, and single nucleotide modifications (e.g., point mutations and single nucleotide polymorphisms (SNPs). In some cases, a deletion can include the deletion of from about 1 to about 100 nucleotides (e.g., from about 1 to about 90, from about 1 to about 80, from about 1 to about 70, from about 1 to about 60, from about 1 to about 50, from about 1 to about 40, from about 1 to about 30, from about 1 to about 20, from about 1 to about 10, from about 1 to about 5, from about 5 to about 100, from about 25 to about 100, from about 50 to about 100, from about 75 to about 100, from about 2 to about 75, from about 3 to about 50, from about 7 to about 40, from about 10 to about 30, from about 12 to about 25, from about 2 to about 10, from about 10 to about 20, from about 20 to about 30, from about 30 to about 40, or from about 40 to about 50 nucleotides). For example, a deletion can include the deletion of 3 nucleotides. In some cases, a insertion can include the insertion of from about 1 to about 100 nucleotides (e.g., from about 1 to about 90, from about 1 to about 80, from about 1 to about 70, from about 1 to about 60, from about 1 to about 50, from about 1 to about 40, from about 1 to about 30, from about 1 to about 20, from about 1 to about 10, from about 1 to about 5, from about 5 to about 100, from about 25 to about 100, from about 50 to about 100, from about 75 to about 100, from about 2 to about 75, from about 3 to about 50, from about 7 to about 40, from about 10 to about 30, from about 12 to about 25, from about 2 to about 10, from about 10 to about 20, from about 20 to about 30, from about 30 to about 40, or from about 40 to about 50 nucleotides). In cases where a nucleic acid sequence includes one or more mutations, the mutations can be disease-causing mutations. For example, a disease causing mutation can be deletion in CFTR (e.g., a three nucleotide deletion in the CFTR gene that causes a deletion of a phenylalanine residue at position of 508 of the CFTR polypeptide (ΔF508)) that causes cystic fibrosis.

This document also provides methods for treating a mammal having a genetic disease or genetic condition (e.g., a monogenetic disease or monogenetic condition) caused, at least in part, by one or more mutations in a nucleic acid sequence (e.g., a coding sequence such as a gene). For example, editing (e.g., correcting) one or more mutations in a nucleic acid sequence (e.g., one or more mutations in a gene associated with a genetic disease or genetic condition) can be effective to treat a mammal having a genetic disease or genetic condition. For example, methods for treating a mammal having a genetic disease or genetic condition can include contacting a cell of the mammal (e.g., a cell obtained from the mammal and/or a cell within the mammal) with a fusion protein described herein (e.g., a fusion protein containing a RecA polypeptide and a CPP) and a single stranded oligonucleotide described herein (e.g., single stranded oligonucleotide including a corrected nucleic acid sequence and capable of hybridizing to a target sequence).

A mammal having any appropriate genetic disease or genetic condition can be treated as described herein. In some cases, a genetic disease or genetic condition can be a monogenetic disease or monogenetic condition. Examples of monogenetic diseases and monogenetic conditions include, without limitation, color blindness, cystic fibrosis, haemochromatosis, haemophilia, phenylketonuria, polycystic kidney disease, sickle-cell disease, Tay-Sachs disease, Huntington's disease, Marfan syndrome, Duchene muscular dystrophy, and some cancers.

Any appropriate mammal (e.g., humans, non-human primates, monkeys, bovine species, pigs, horses, dogs, cats, sheep, goat, and rodents) having a monogenetic disease or monogenetic condition can be treated as described herein. In some cases, humans can be treated using the methods and materials provided herein. For example, a human having, or at risk of developing (e.g., based, at least in part, on the present of a disease-causing mutation in one or more cells within the human), cystic fibrosis can be treated by using the methods and materials provided herein to correct a CFTR coding sequence in one or more cells within the human. For example, a human having, or at risk of developing (e.g., based, at least in part, on the present of a disease-causing mutation in one or more cells within the human), Duchene muscular dystrophy can be treated by using the methods and materials provided herein to correct a dystrophin coding sequence in one or more cells within the human.

Any appropriate method can be used to deliver one or more nucleoprotein filaments described herein (e.g., nucleoprotein filaments including a fusion protein described herein and a single stranded oligonucleotide) to a cell (e.g., to a cell in a mammal).

In some cases, the methods and materials provided herein also can be used in other organisms. For example, the methods and materials provided herein can be used in plant cells, fungal cells, and/or bacterial cells.

The invention will be further described in the following examples, which do not limit the scope of the invention described in the claims.

### EXAMPLES

### Example 1. Materials

Most of the chemicals were purchased from Sigma; DMEM/F-12 medium and fetal bovine serum were derived from Thermo Scientific; Restriction endonucleases, from New England Biolabs; QuikChange site-directed mutagenesis kit, from Stratagene; Anti-mouse Ig conjugated with horseradish peroxidase, from Amersham Biosciences; Chemiluminescent substrates for western blotting, from Pierce; RecA DNA (pDONR221.RecA), from DNASU.

### Example 2. RecA-CPP Fusion Nucleic Acid Construct

In order to express the RecA-CPP fusion protein in mammalian cells, the 5′ part of the RecA DNA (pDONR221.RecA was used as template) was amplified by using the primers NutRecAfwasu and RecA324rvasu (Table 1); the fusion part between RecA and GFP was performed by two steps PCR, i.e., the 1^{st} piece (pDONR221.RecA was used as template) was amplified by using RecA763fwasu and RecAlinkgfprvasu (Table 1), whereas the 2^{nd} part (pCDH-CMV-MCS-EF1-copGFP was used as template) was amplified by using RecAlinkgfpfwasu and CDHGFP6658rv (Table 1); upon amplification of these two pieces DNA, they were used as templates to put them together by using RecA763fwasu and CDHGFP6658rv (Table 1) as primers; the 3′ part of the fusion gene was amplified by three steps, i.e., the 1^{st} piece (pCDH-CMV-MCS-EF1-copGFP was used as template) was amplified by using Gfp6302rv and 1^{st}.CPPry (Table 1) as primers; the 2^{nd} part (the 1^{st} piece of the PCR product was used as template) was amplified by using Gfp6302rv and 2^{nd} CPPrv (Table 1) as primers; whereas the 3^{rd} part (the 2nd part of the PCR product was used as template) was amplified by using Gfp6302rv and 3rd.CPPrv (Table 1) as primers. All these pieces of PCR products were cloned into pBluescript and sequenced completely to make sure that there is no mutation occurred in the clones. Two bigger pieces, i.e., the N-terminal half (cloned by combining the XmaI-DraIII fragment from the 1^{st} PCR clone, the DraIII-AseI fragment from pDONR221.RecA and the AseI-HindIII fragment from the RecA.GFP fusion clone) and C-terminal half (cloned by combining the HindIII-ApaL1 fragment from the RecA.GFP fusion clone, the ApaL1-BglI fragment from pCDH-CMV-MCS-EF1-copGFP and the Bgl1-HindIII fragment from the 3^{rd} part of the clone), were cloned into pBluescript and sequenced completely. The N-terminal half and C-terminal half clones were used to make full length fusion gene in pNUT vector (see, e.g., Palmiter et al., 1987 Cell 50: 435-443). In order to make a shorter version of the fusion protein, the two primers, rmgfpbamhlfw and rmgfpbamhlry (Table 1), were used to delete the GFP gene from the full length fusion gene by employing the QuikChange Site-directed Mutagenesis kit (Stratagene). The longer version of the fusion gene (named as pNUT-RecA-GFP-CPP) and the shorter version of the fusion gene (named as pNUT-RecA-CPP) were sequenced completely to make sure that there is no mutation occurred in the final clones.

In order to express the RecA-CPP fusion proteins in bacteria, the two primers, ET32RecAfw1step and ET32RecArv1step (Table 1), were used to modify the 5′ part of the N-terminal half clone by employing the QuikChange Site-directed Mutagenesis kit. The modified N-terminal half clone and the original C-terminal half clone were used to make full length fusion gene in pET32a expression vector. In order to make shorter version of the fusion protein, the two primers, rmgfpbamh1fw and rmgfpbamh1rv (Table 1), were used to delete the GFP gene from the full length fusion gene. The longer version of the fusion gene (named as pET32a. RecA-GFP-CPP) and the shorter version of the fusion gene (named as pET32a-RecA-CPP) were sequenced completely to make sure that there is no mutation occurred in the final clones.

TABLE 1
List of Oligonucleotides

| SEQ | | |
|---|---|---|
| ID | | |
| NO | Name | Sequence |
| 14 | NutRecAfwasu | GCCCGGGACCATGGCTATTGATGAGAATAAAC |
| 15 | ET32RecAfwasu | CTCTAGAAATAATTTTGTTTAACTTTAAGAAGGAGATATACATATGGCTATTGATGAGAATAAAC |
| 16 | RecA324rvasu | CAATTTCTTGGCATAGATTGG |
| 17 | RecA763fwasu | CCATTCAAACAAGCTGAATTC |
| 18 | RecAlinkGfpfwasu | GAAACCAACGAAGAATTTAGTGGCCTACGATCGCGAGCAGCTGCGAACACGATGAGTATTCAACATTTC |
| 19 | CDHGFP6658rv | CGGGATAATACCGCGCCAC |
| 20 | RecAlinkGfprvasu | GAAATGTTGAATACTCATCGTGTTCGCAGCTGCTCGCGATCGTAGGCCACTAAATTCTTCGTTGGTTTC |
| 21 | Gfp6302rv | GCTTCCCGGCAACAATTAATAG |
| 22 | Gfp6019fw | GAGTAAACTTGGTCTGACAG |
| 23 | 1^{st}.CPPrv | GTGAAGTTGACATCCAAAAAGGATGTTTTCTCGTGCTGCAGCCCAATGCTTAATCAGTGA |
| 24 | 2^{nd}.CPPrv | CTTCTTCCCTGCCGTAATGGTGATGGTGATGGTGATGGTGATGGTGAAGTTGACATCCAAA |
| 25 | 3^{rd}.CPPrv | GCGGCCGCCTATCTTCGTCGCTGTCTCCGCTTCTTCCTGCCGTAATG |
| 26 | ET32RecAfw1step | GGTGGCGGCCGCTCTAGAAATAATTTTGTTTAACTTTAAGAAGGAGATATACATATGGCTATTGATGAGAA |
| | | TAAAC |
| 27 | ET32RecArv1step | GTTTATTCTCATCAATAGCCATATGTATATCTCCTTCTTAAAGTTAAACAAAATT |
| | | ATTTCTAGACGGCCGCCACC |
| 28 | rmgfpbamh1fw | CGAGCAGCTGCGAACACGGGATCCGCTGCAGCACGAGAAAAC |
| 29 | rmgfpbamh1rv | GTTTTCTCGTGCTGCAGCGGATCCCGTGTTCGCAGCTGCTCG |

Two versions of the RecA-CPP fusion proteins, i.e., shorter version (RecA-CPP) and longer version (RecA-GFP-CPP), were designed (FIG. 1). RecA-CPP contains: 1) RecA; 2) an L1 linker (see, e.g., Orban et al., 2008 Biochem Biophys Res Commun 367:667-673; and Hou et al., 2009 Biochemistry 48:9122-9131); 3) Tag1, the epitope of the MRP1 mAb 42.4 (see, e.g., Hou et al., 2000 J Biol Chem 275:20280-20287); 4) Tag2, a ten histidine residue tag; 5) CPP, a cell-penetrating-peptide, i.e., transactivator of transcription (TAT) peptide (see, e.g., Frankel et al., 1988 Cell 55:1189-1193; Green et al., 1988 Cell 55:1179-1188; Debaisieux et al., 2012 Traffic 13:355-363; Schwarze et al., 2000 Trends in cell biology 10:290-295; and Dietz et al., 2004 Molecular and cellular neurosciences 27:85-131). The longer version, i.e., RecA-GFP-CPP, contains: 1) RecA; 2) L1; 3) GFP; 4) L2, a two-alanine residue short linker; 5) Tag1; 6) Tag2; and 7) CPP.

### Example 3. Cell Culture and Transfection

Baby hamster kidney (BHK) cells were grown in DMEM/F-12 medium containing 5% fetal bovine serum at 37° C. in 5% CO2. Subconfluent cells were transfected with plasmid DNAs containing either longer version of the fusion gene (pNUT-RecA-GFP-CPP) or shorter version of the fusion gene (pNUT-RecA-CPP) in the presence of 20 mM HEPES (pH 7.05), 137 mM NaCl, 5 mM KCl, 0.7 mM Na₂HPO₄, 6 mM dextrose and 125 mM CaCl₂ (see, e.g., Chang et al., 1997 J Biol Chem 272:30962-30968). Whole mixture of the methotrexate-resistant cells was used to determine the expression of the fusion proteins with the MRP1 monoclonal antibody (mAb) 42.4 (see, e.g., Hou et al., 2000 J Biol Chem 275:20280-20287).

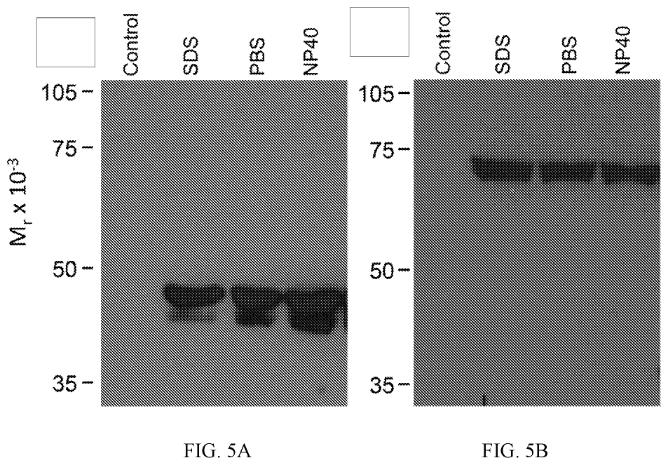

In order to express these fusion proteins in mammalian cells, the two fusion genes diagramed in FIG. 1 were inserted into a mammalian expression vector, i.e., pNUT (see, e.g., Palmiter et al., 1987 Cell 50:435-443). Upon transformation of BHK cells with these two constructs, i.e., pNUT-RecA-CPP and pNUT-RecA-GFP-CPP, the methotrexate resistant cells were used to determine the expression of these fusion proteins. The results in FIG. 5A clearly indicated that RecA-CPP fusion protein is expressed in BHK cells. In addition, the amount of the fusion protein in cells lysed with SDS is similar to the cells lysed with NP40 or lysed in PBS, suggesting that majority of the fusion protein expressed in BHK cells is in soluble fraction. The expression of the longer version, i.e., RecA-GFP-CPP, in BHK cells is similar to the shorter version (FIG. 5B).

Expression of fusion proteins in the cells was examined using western blotting as described in Example 5.

### Example 4. Expression of the RecA-CPP Fusion Proteins in Prokaryotic DL21 Cells

The DL21 competent cells were transformed with either pET32a-RecA-GFP-CPP or pET32a-RecA-CPP. The freshly received ampicillin-resistant colonies were used to inoculate 1 mL of 50% Luria-Bertani Broth (LB) and 50% super LB (with 100 μg/mL ampicillin) and cells were grown at 37° C. for ˜6 hours. 10-100 μL (depending on the cell density) of these bacteria were used to inoculate 100 mL of 50% LB and 50% super LB (with 100 μg/mL ampicillin) and the cells were grown overnight at 16° C. until the OD600 reaching 0.6-1.0. After adjusting temperature to 4° C., isopropyl β-D-1-thiogalactopyranoside (IPTG) was added to 1 mM (final concentration) and cells were grown at this temperature for 16 hours. The cells were harvested by centrifugation at 5,000×g for 5 minutes at 4° C. and the pellets and supernatants were used to determine the expression of these fusion proteins.

Expression of fusion proteins in the cells was examined using western blotting as described in Example 5.

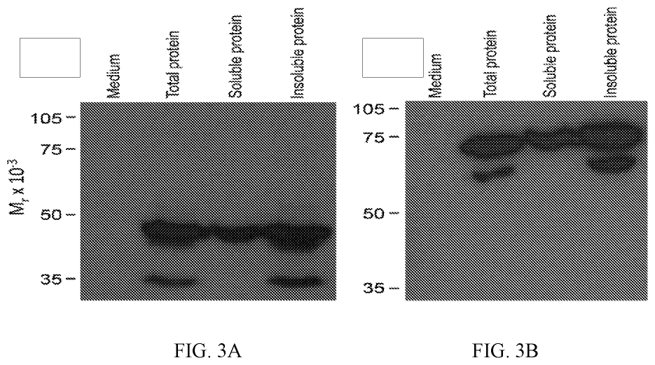

Expression of the fusion proteins completely blocked DL21 cell growth: The results in FIG. 3A indicated that RecA-CPP fusion protein is clearly expressed in DL21 cells. The protein expressed in DL21 cells is not leaked out to the medium and it also clearly indicated that certain amount the fusion protein is in soluble fraction. The expression of the longer version, i.e., RecA-GFP-CPP, in DL21 cells is similar to the shorter version (FIG. 3B).

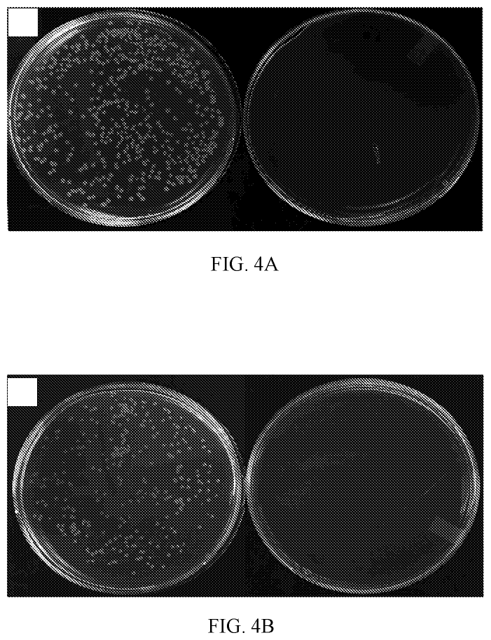

In order to test whether the expression of the fusion proteins has effect on cell growth or not, the DL21 competent cells transformed with either pET32a-RecA-CPP or pET32a. RecA-GFP-CPP were plated out on the plates containing either 100 μg/mL ampicillin or 100 μg/mL ampicillin and 0.25 mM IPTG. Interestingly, regardless whether the shorter version or the longer version of the fusion constructs were used, the cells plated out on the plates containing only 100 μg/mL ampicillin grow very well, whereas the cells plated out on the plates containing 100 μg/mL ampicillin and 0.25 mM IPTG did not form visible colonies (FIGS. 4A and 4B), implying that IPTG induction of the fusion proteins significantly inhibited prokaryotic cell growth.

### Example 5. Identification of RecA-CPP Fusion Proteins

Western blot was performed according to the routine protocol. For RecA-CPP fusion proteins expressed in BL21 cells, the following four samples were prepared: 1) the proteins in medium (the proteins in medium were precipitated with trichloroacetic acid and the pellets were dissolved in 1× sample buffer containing 1× protease inhibitor cocktail, i.e., Aprotonin, 2 μg/mL; Benzamide, 121 μg/mL; E64, 3.5 μg/mL; Leupeptin, 1 μg/mL; and Pefabloc, 50 μg/mL); 2) total proteins in bacteria [Cell pellets were re-suspended in 1× nickel bead binding buffer (20 mM Tris/HCl, pH7.9; 500 mM NaCl) containing 10% glycerol, 1× protease inhibitors and 20,000 units/mL of lysozyme, incubated at 37° C. for 15 minutes, added sodium dodecyl sulfate (SDS) to 2% (final concentration) and then sonicated for 20 bursts to break the DNA]; 3) proteins in soluble fraction (Cell pellets were re-suspended in 1× nickel bead binding buffer containing 10% glycerol, 1× protease inhibitors and 20,000 units/mL of lysozyme, incubated at 37° C. for 15 minutes, and then sonicated for 20 bursts to break the DNA. The soluble fraction was collected after centrifugation at 14,000 RPM for 10 minutes); 4) proteins in insoluble fraction (the pellets derived from previous step were dissolved in 1× nickel bead binding buffer containing 10% glycerol, 1× protease inhibitors and 2% SDS and then sonicated for 20 bursts to break the DNA).

For RecA-CPP fusion proteins expressed in BHK cells, the following three samples were prepared: 1) Cells lysed with SDS and sonication [Cells were lysed with phosphate buffered saline (PBS) containing 1× protease inhibitors and 2% SDS and then sonicated for 20 bursts to break the DNA]; 2) Cells lysed with sonication (Cells re-suspended in PBS containing 1× protease inhibitors were sonicated for 20 bursts to break the DNA); 3) Cells lysed with NP40 buffer [Cells were lysed with NP40 cell lysis buffer (0.1% NP40, 150 mM NaCl, 50 mM Tris, 10 mM Sodium Molybdate, pH 7.6) containing 1× protease inhibitors by shaking the plates in cold room for 30 minutes. The supernatants were collected after centrifugation at 14,000 RPM].

Samples were subjected to SDS-PAGE, followed by transferring the proteins to nitrocellulose membranes, probed with the MRP1 primary antibody 42.4 (see, e.g., Hou et al., 2000 J Biol Chem 275:20280-20287) overnight at 4° C., washed with PBS containing 0.1% Tween-20 and then incubated with anti-mouse Ig conjugated with horse radish peroxidase. Chemiluminescent film detection was performed according to the manufacturer's recommendations (Pierce).

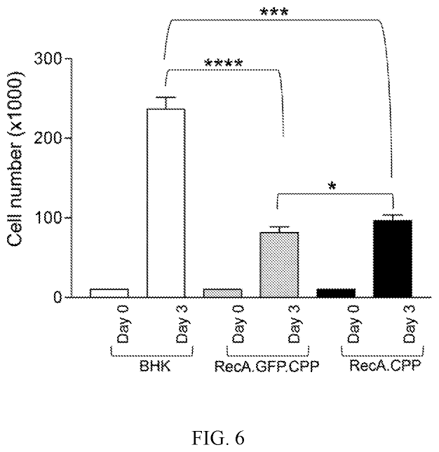

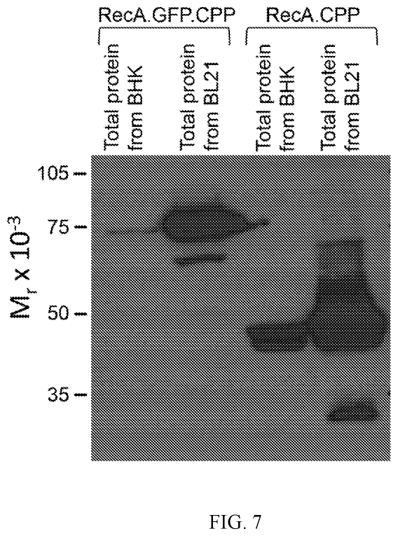

Statistical Analysis: The results in FIG. 6 were presented as means±SD from the triplicate experiments. The two-tailed P values were calculated based on the unpaired t-test from GraphPad Software Quick Calcs. By conventional criteria, if P value is less than 0.05, the difference between two samples is considered to be statistically significant.

Expression of the fusion proteins significantly inhibited BHK cell growth: In order to test whether the expression of the fusion proteins has effect on mammalian cell growth or not, 10,000 BHK cells expressing either RecA-CPP or RecA-GFP-CPP were plated out on day 0 and counted on day 3. Interestingly, the number of BHK cells expressing RecA-CPP is similar to the cells expressing RecA-GFP-CPP, whereas the number of parental BHK cells is significantly higher than either cells expressing RecA-CPP or RecA-GFP-CPP (FIG. 6), suggesting that expression of these fusion proteins significantly inhibited mammalian cell growth.

### Example 6. Gene Editing Using the Nucleoprotein Filament

Ability of the present nucleoprotein filaments comprising fusion proteins and single stranded nucleotide to correct a mutation in a cell or a subject is studied in this example.

In one embodiment, CF ΔF508 mutation cell lines (i.e., cells containing a deletion of three nucleotides coding for phenylalanine at position of 508 (ΔF508) in CFTR) or cell lines having a mutation in the ATP-binding cassette transporter C7 (ABCC7) gene are treated with a fusion protein comprising a RecA, a CPP and a single stranded nucleotide comprising a sequence that is sufficiently complementary to the target sequence and a corrected sequence to correct the mutations.

In one embodiment, an animal model carrying a CF ΔF508 mutation is treated with the present nucleoprotein filament.

In one embodiment, a human having one or more cells having a CF ΔF508 mutation (i.e., cells containing a deletion of three nucleotides coding for phenylalanine at position of 508 (ΔF508) in CFTR are treated with nucleoprotein filaments containing one or more fusion proteins including a RecA polypeptide and a CPP, and a single stranded oligonucleotide comprising a sequence that is sufficiently complementary to the target sequence and a corrected sequence to correct the mutations.

### Example 7. Model Systems to Test Frequencies of Homology-Directed Recombination (HDR)

A model system was established to test the efficiency of HDR in eukaryotic cells. A dual marker system in one construct was designed in which the expression of mouse DHFR in eukaryotic cells provides methotrexate resistance whereas the expression of GFP generates green cells. The construct was based on a dual promoter system in pNUT expression vector as described elsewhere (see, e.g., Palmiter et al., 1987 Cell 50:435-443). This system can be designed to express any appropriate polypeptide, such as MRP1 or CFTR.

### GFP Model

When a cell expresses a wild-type DHFR and a methotrexate resistance phenotype, GFP is used to detect, and optionally evaluate, HDR.

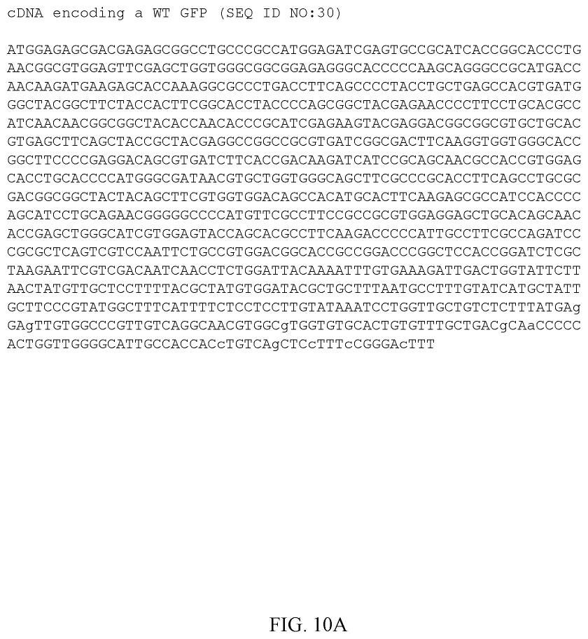

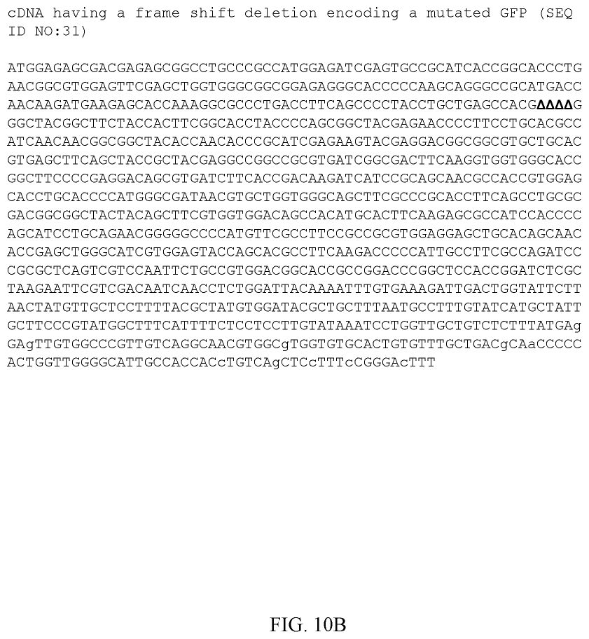

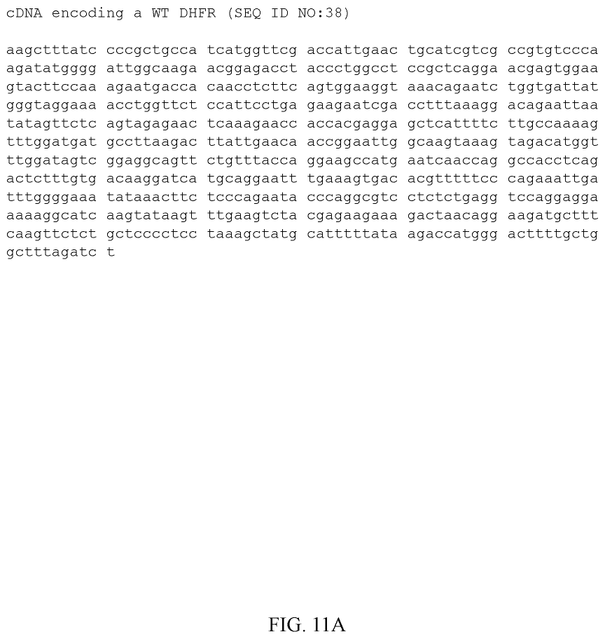

A construct including a cDNA encoding a wild type DHFR and a cDNA encoding a loss-of-function mutated GFP is used. A deletion of nucleotides TGAT from 185 to 188 of a GFP cDNA generates a frame-shift mutation that leads to expression of a mutated GFP polypeptide and loss of function. An exemplary nucleotide sequence of wild-type GFP cDNA (SEQ ID NO:30) is shown in FIG. 10A, and an exemplary nucleotide sequence of the frame-shift deletion mutated GFP (SEQ ID NO:31) is shown in FIG. 10B.

Insertion of these 4 nucleotides via HDR using single stranded oligonucleotides corrects the deletion and restores GFP expression and function in the cell to provide a fluorescent phenotype. Exemplary single stranded oligonucleotides that can correct the deletion shown in FIG. 10B are as set forth in Table 2.

Counting of the green cells (e.g., by fluorescence activated cell sorting (FACS)) is used to determine the efficiency of HDR mediated by ssDNA-RecA-CPP. This model can also be used to determine the efficiency of HDR by other gene editing systems such as ZFNs, TALENs, and CRISPR/Cas9.

TABLE 2
Single strand oligonucleotides used to
correct the GFP cDNA frame-shift deletion
shown in FIG. 10B (highlighted letters
are the nucleotides inserted
to correct the deletion mutation).

| SEQ | | |
|---|---|---|
| ID | | |
| NO: | Name | Sequence |
| 32 | CR.TGAT.fw1 | GGCGCCCTGACCTTCAGCCCCTACCTGCTGAGCCA |
| | | CGTGATGGGCTACGGCTTCTACCAC |
| 33 | CR.TGAT.fw2 | AACAAGATGAAGAGCACCAAAGGCGCCCTGACCTT |
| | | CAGCCCCTACCTGCTGAGCCACGTGATGGGCTACG |
| | | GCTTCTACCAC |
| 34 | CR.TGAT.fw3 | CCCAAGCAGGGCCGCATGACCAACAAGATGAAGAG |
| | | CACCAAAGGCGCCCTGACCTTCAGCCCCTACCTGC |
| | | TGAGCCACGTGATGGGCTACGGCTTCTACCAC |
| 35 | CR.TGAT.rv1 | ccgct ggggt aggtg ccgaa gtggt agaag |
| | | ccgta gccc ATCA CGTG GCTC AGCA GGTA |
| | | G |
| 36 | CR.TGAT.rv2 | t gcagg aaggg gttct cgtag ccgct |
| | | ggggt aggtg ccgaa gtggt agaag ccgta |
| | | gccc ATCA CGTG GCTC AGCA GGTA G |
| 37 | CR.TGAT.rv3 | tagcc gccgt tgttg atggc gt gcagg |
| | | aaggg gttct cgtag ccgct ggggt aggtg |
| | | ccgaa gtggt agaag ccgta gccc ATCA |
| | | CGTG GCTC AGCA GGTA G |

### DHFR Model

When a cell expresses a wild-type GFP, DHFR is used to detect, and optionally evaluate, HDR.

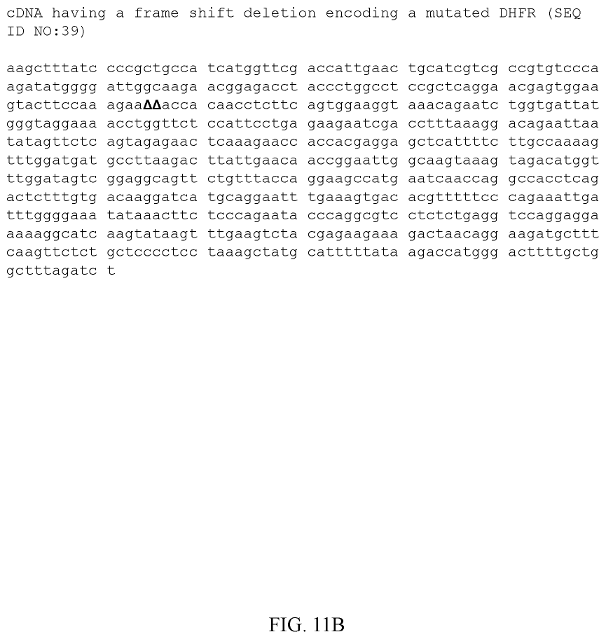

A construct including a cDNA encoding a wild type GFP and a cDNA encoding a loss-of-function mutated DHFR is used. A deletion of nucleotides TG from 135 to 136 of a DHFR cDNA generates a frame-shift mutation that leads to expression of a mutated DHFR polypeptide and loss of function. An exemplary nucleotide sequence of wild-type DHFR cDNA (SEQ ID NO:38) is shown in FIG. 11A, and an exemplary nucleotide sequence of the frame-shift deletion mutated DHFR (SEQ ID NO:39) is shown in FIG. 11B.

Insertion of these 2 nucleotides via HDR using single stranded oligonucleotides corrects the deletion and restores DHFR expression and function in the cell to provide a methotrexate resistance phenotype. Exemplary single stranded oligonucleotides that can correct the deletion shown in FIG. 11B are as set forth in Table 3.

Counting of methotrexate resistant colonies (e.g., compared to cells without methotrexate treatment) is used to determine the efficiency of HDR mediated by ssDNA-RecA-CPP nucleoprotein filaments. This model can also be used to determine the efficiency of HDR mediated by other gene editing systems such as ZFNs, TALENs, and CRISPR/Cas9.

TABLE 3
Single strand oligonucleotides used to correct
the DHFR cDNA frame-shift deletion shown in FIG.
11B (highlighted letters are the nucleotides
inserted to correct the deletion mutation).

| SEQ | | |
|---|---|---|
| ID | | |
| NO: | Name | Sequence |
| 40 | CrdeTGinDHFRfw1 | tggcct ccgctcagga acgagtggaa |
| | | gtacttccaa agaatgacca |
| | | caacctcttc agtg |
| 41 | CrdeTGinDHFRfw2 | ggcaaga acggagacct accctggcct |
| | | ccgctcagga acgagtggaa |
| | | gtacttccaa agaatgacca |
| | | caacctcttc agtg |
| 42 | CrdeTGinDHFRfw3 | gtgtccca agatatgggg attggcaaga |
| | | acggagacct accctggcct |
| | | ccgctcagga acgagtggaa |
| | | gtacttccaa agaatgacca |
| | | caacctcttc agtg |
| 43 | CrdeTGinDHFRry1 | TCACCA CATTCTGTTT ACCTTCCACT |
| | | GAAGAGGTTG TGGTCATTCT |
| | | TTGGAAGTAC TTCC |
| 44 | CrdeTGinDHFRry2 | ACCAGGT TTTCCTACCC ATAATCACCA |
| | | CATTCTGTTT ACCTTCCACT |
| | | GAAGAGGTTG TGGTCATTCT |
| | | TTGGAAGTAC TTCC |
| 45 | CrdeTGinDHFRry3 | GATTCTTC TCAGGAATGG AGAACCAGGT |
| | | TTTCCTACCC ATAATCACCA |
| | | CATTCTGTTT ACCTTCCACT |
| | | GAAGAGGTTG TGGTCATTCT |
| | | TTGGAAGTAC TTCC |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A fusion protein comprising a RecA polypeptide and cell penetrating peptide (CPP).

2. The fusion protein of claim 1, wherein said RecA polypeptide comprises an amino acid sequence set forth in SEQ ID NO:1, SEQ ID NO:2, SEQ ID NO:3, or SEQ ID NO:4.

3. (canceled)

4. The fusion protein of claim 1, wherein said CPP is selected from the group consisting of a trans-activating transcriptional activator (TAT) peptide sequence, a Pep-1 peptide sequence, and a MPG peptide sequence.

5. The fusion protein of claim 4, wherein said CPP is a TAT peptide, and wherein said TAT peptide sequence comprises the amino acid sequence YGRKKRRQRRR (SEQ ID NO:5).

6. The fusion protein of claim 4, wherein said CPP is a Pep-1 peptide, and wherein said Pep-1 peptide sequence comprises the amino acid sequence KETWWETWWTEWSQPKKKRKV (SEQ ID NO:6).

7. The fusion protein of claim 4, wherein said CPP is a MPG peptide, and wherein said MPG peptide sequence comprises the amino acid sequence SVVDRVAEQDTQA (SEQ ID NO:7).

8. (canceled)

9. The fusion protein of claim 1, said fusion protein further comprising a peptide linker present between said RecA polypeptide and said CPP.

10. The fusion protein of claim 9, wherein said peptide linker is selected from the group consisting of a peptide sequence including SGLRSRAAANT (SEQ ID NO:8), one or more alanine residues, one or more glycine residues, and combinations thereof.

11. The fusion protein of claim 1, said fusion protein further comprising a peptide tag, wherein said peptide tag is an antibody epitope or a fluorescent protein.

12. (canceled)

13. The fusion protein of claim 11, wherein said antibody epitope is a multidrug resistance protein 1 (MRP1) antibody epitope.

14. (canceled)

15. The fusion protein of claim 11, wherein said fluorescent protein is a green fluorescent protein.

16. The fusion protein of claim 11, said fusion protein comprising an antibody epitope or a fluorescent protein, wherein said antibody epitope is a MRP1 antibody epitope, and wherein said fluorescent protein is a green fluorescent protein.

17-20. (canceled)

21. A nucleic acid construct encoding the fusion protein of claim 1.

22-25. (canceled)

26. A nucleoprotein filament comprising:
one or more fusion proteins of claim 1; and
a single stranded oligonucleotide, wherein said single stranded oligonucleotide can hybridize to a target sequence having one or more mutations, and wherein said single stranded oligonucleotide comprises a corrected nucleic acid sequence.

27. A method for editing the genome of a cell, said method comprising:
contacting the cell with a) a fusion protein comprising a RecA polypeptide and cell penetrating peptide (CPP); and b) a single stranded oligonucleotide, wherein said single stranded oligonucleotide can hybridize to a target sequence having one or more mutations, and wherein said single stranded oligonucleotide comprises a corrected nucleic acid sequence.

28. The method of claim 27, wherein said cell is a prokaryotic cell.

29. The method of claim 27, wherein said cell is a eukaryotic cell.

30. (canceled)

31. A method for treating a mammal having a monogenetic disease, the method comprising:
contacting a cell in the mammal with a) a fusion protein comprising a RecA polypeptide and cell penetrating peptide; and b) a single stranded oligonucleotide, wherein said single stranded oligonucleotide can hybridize to a target sequence in a genome within said cell, wherein said target sequence comprises a nucleic acid sequence comprising one or more disease-causing mutations, and wherein said single stranded oligonucleotide comprises a corrected nucleic sequence.

32. The method of claim 31, wherein said mammal is a human.

33. The method of claim 31, wherein said monogenetic disease is selected from the group consisting of color blindness, cystic fibrosis, haemochromatosis, haemophilia, phenylketonuria, polycystic kidney disease, Tay-Sachs disease, Huntington's disease, Marfan syndrome, sickle-cell disease, duchenne muscular dystrophy, and cancer.

34-43. (canceled)
